(19) United States
(12) Patent Application Publication
(10) Pub. No.: US 2008/0199469 A1
(43) Pub. Date: Aug. 21, 2008

(54) Regulation and function of TPL-2

(75) Inventor: Steven Ley, London (GB)

Correspondence Address: Edwards Angell Palmer & Dodge LLP, 111 HUNTINGTON AVENUE, BOSTON, MA, 02199, US

(73) Assignee: Medical Research Council

(21) Appl. No.: 11/441,507

(22) Filed: May 26, 2006

Related U.S. Application Data
(63) Continuation of application No. PCT/GB04/05021, filed on Nov. 29, 2004.
(60) Provisional application No. 60/527,087, filed on Dec. 3, 2003.

(30) Foreign Application Priority Data
Nov. 28, 2003 (GB) 0327707.6

(51) Int. Cl. A61K 39/395; G01N 33/00; G01N 33/566; G01N 33/567; C12Q 1/48; A61P 43/00; G06G 7/48; C07K 16/18; C07K 1/00; C12N 5/06

(52) U.S. Cl. 424/138.1; 436/86; 436/501; 435/7.21; 435/15; 530/387.7; 530/300; 435/375; 703/11; 530/402

(57) ABSTRACT

The invention relates to the use of ABIN2 to stabilise TPL-2, and a ternary complex formed between ABIN2, TPL-2 and p105, as well as assays for compounds capable of modulating the interaction between ABIN2 and TPL-2 and/or p105 and use of such compounds in the treatment of inflammatory conditions.

### RELATED APPLICATIONS

This application is a continuation application which claims priority to PCT application Serial No. PCT/GB2004/005021, filed Nov. 29, 2004, which claims priority to U.S. Provisional Application 60/527,087 filed Dec. 3, 2003 and Great Britain Application Serial No. 0327707.6 filed Nov. 28, 2003, the entireties of which are incorporated herein by reference.

### FIELD OF THE INVENTION

The present invention relates to the regulation and function of TPL-2 activity. In particular, the invention relates to regulation of the interaction of TPL-2 with ABIN2 and p 105.

### BACKGROUND TO THE INVENTION

The present invention relates to the regulation of a signalling pathway. In particular, the invention relates to the modulation of the regulation of the ERK/MAP Kinase pathway by TPL-2 through an upstream regulator, ABIN2. Moreover, the invention relates to the use of ABIN2 as a target for the development of agents capable of modulating TPL-2 and p105 and especially agents capable of modulating the interaction of the IκB p105 and the kinase TPL-2 in the ERK/MAP Kinase cascade.

Mammals express five NF-κB proteins: RelA, RelB, c-Rel, NF-κB1 p50 and NF-κB2 p52 which bind DNA as homo- and heterodimers and play an essential role in coordinating the transcription of genes involved in the innate and adaptive immune responses (10). NF-κB dimers are retained in the cytoplasm of unstimulated cells through their interaction with a family of inhibitory proteins, termed IκBs, which includes IκBα, IκBβ, IκBε and NF-κB1 p105 (the precursor of p50). NF-κB agonists which trigger the canonical NF-κB signalling pathway, such as tumour necrosis factor (TNF) α and lipopolysaccharide (LPS), induce IκB phosphorylation by the IκB kinase (IKK) complex. This promotes IκB ubiquitination and subsequent proteolysis by the 26S proteasome (17). Associated NF-κB dimers are thereby released to translocate into the nucleus and modulate gene expression.

Although p105 has been shown to associate with p50, c-Rel and RelA in the cytoplasm (20, 21), genetic studies in mice have revealed that p105 is only essential for correct regulation of the nuclear translocation of p50 homodimers (15). p50 is produced by proteasome-mediated proteolysis of p105, which occurs in a constitutive, unregulated fashion (17). However, following cellular stimulation with ligands such as TNFα, two serines in the p105 PEST domain are rapidly phosphorylated by the IKK complex which triggers complete p105 degradation with little detectable effect on processing to p50 (18, 22). This results in the release of associated p50, and other Rel subunits, which can then translocate into the nucleus. Analysis of knockout mice that lack the C-terminal (IκB-like) half of p105 whilst still expressing p50 has suggested a role for p105 in the regulation of cytokines involved in inflammatory responses in both T cells and macrophages (15).

Lipopolysaccharide (LPS) stimulation of TLR4 on macrophages induces the production of proinflammatory cytokines as part of the innate immune response to gram-negative bacterial infection (25). LPS triggering of proinflammatory cytokine gene expression involves the activation of both signalling pathways that regulate NF-κB and all of the major MAP kinase (MAP K) subtypes (extracellular signal-related kinases (ERK)-1/2, Jun amino-terminal kinases (JNK) and p38 (14, 31)). MAP K activation involves three-tiered kinase cascades in which MAP Ks are activated by MAP K kinases (MAP 2-K), which in turn are activated by MAP K kinase kinases (MAP 3-K) (6).

TPL-2 was originally identified, in a C-terminally deleted form, as the product of an oncogene associated with Moloney murine leukaemia virus-induced T cell lymphomas in rats (Patriotis, et al., (1993) Proc. Natl. Acad. Sci. USA 90:2251-2255). TPL-2 is a protein serine kinase which is homologous to MAP kinase kinase kinases (3K) in its catalytic domain (Salmeron, A., et al., (1996) EMBO J. 15:817-826) and is >90% identical to the proto-oncogene product of human COT (Aoki, M., (1993) et al. J. Biol. Chem. 268:22723-22732). TPL-2 is also highly homologous to the kinase NIK, which has been shown to regulate the inducible degradation of IκB-α (Malinin et al., (1997) Nature 385:540-544; WO 97/37016; May and Ghosh, (1998) Immunol. Today 19:80-88).

In macrophages, LPS activation of MEK-1/2, the MAP 2-Ks which phosphorylate and activate ERK-1/2, is mediated by TPL-2 MAP 3-kinase (9). Recent research has revealed an unexpected novel function for NF-κB1 p105 in regulating this signalling pathway (29). The C-terminal half of p105 forms a high affinity, stoichiometric association with the TPL-2 MEK kinase (2, 3). Interaction with p105 is required for stabilization of TPL-2 protein and in p105-deficient macrophages the steady-state levels of TPL-2 protein are very low (2, 29). As a result, LPS activation of MEK is severely reduced in these cells (29). It has also been shown that the interaction of p105 with TPL-2 negatively regulates its MEK kinase activity by preventing access of MEK to TPL-2 (2). In resting macrophages, in which all of TPL-2 is complexed with p105 (2), TPL-2 MEK kinase activity is therefore actively inhibited. Following LPS stimulation, TPL-2 activation involves its release from p105 (29).

TPL-2 is a drug target studied for effects inter alia in inflammatory disease and oncology. Based on experiments with TPL-2 knockout mice, it is known that TPL-2 is required for septic shock and Crohn's disease (Cell, 103: 1071-1083, 2000; JEM, 196: 1563-1574, 2002). Based on these results is also very likely that TPL-2 is required for the development of rheumatoid arthritis. Methods for the modulation of TPL-2, and the understanding of the biology of TPL-2 activity, are therefore important in the pharmaceutical industry.

### SUMMARY OF THE INVENTION

In order to more fully understand the regulation and function of the TPL-2/p105 complex, we have sought to identify any additional proteins with which it associates. In the present study, affinity purification and peptide mass fingerprinting revealed A20 binding inhibitor of NF-κB (ABIN) 2 (27) as a novel p105-associated protein. A20 gene transcription is induced by a number of stimuli which activate NF-κB (4) and analysis of A20 knockout mice has indicated that A20 regulates the termination of NF-κB activity after TNFα stimulation (19). Overexpression experiments have suggested that ABIN2 may function as a downstream effector of A20 in the inhibition of NF-κB (27). Evidence is presented here that the majority of ABIN2 in macrophages forms a ternary complex with TPL-2 and p105 and that ABIN2 is required for stable TPL-2 protein expression.

In a first aspect of the present invention, therefore, there is provided the method of using an ABIN2 molecule in the stabilisation of TPL-2.

As shown herein, ABIN2 regulates at least post-transcriptional turnover of TPL-2 in vivo and is required for TPL-2 stability. ABIN2 binds to TPL-2, and the portion of the ABIN2 molecule which mediates this binding comprises residues 1-250. More precisely, the binding region can be identified as residing in residues 194-250 of ABIN2.

In a further embodiment, the invention provides a method of using an ABIN2 molecule in the modulation of p105 activity. ABIN2 binds to p105, and the portion of ABIN2 responsible for interaction with p105 is located in amino acids 1-250 of ABIN2.

Advantageously, the invention further provides a method for identifying a compound or compounds capable of modulating the activity of TPL-2, comprising the steps of:
(a) incubating an ABIN2 molecule with the compound or compounds to be assessed; and
(b) identifying those compounds which influence the binding of ABIN2 to TPL-2.

Preferably, the compound or compounds bind to the TPL-2 molecule and/or the ABIN2 molecule.

The complex formed between ABIN2, TPL-2 and p105 is involved in the regulation of the MEK/ERK MAP Kinase signalling pathway, and particularly the stimulation of this pathway by TLR4 in macrophages, which is responsible for inflammatory responses in mammals. Moreover, CD40 and TNFR1 activate ERK via TPL-2 (EMBO J, 22: 3855-3864, 2003). ABIN2 is therefore a drug target for the regulation of this pathway and the treatment of, inter alia, inflammatory conditions.

Accordingly, there is provided a method for identifying a lead compound for a pharmaceutical useful in the treatment of disease, comprising: incubating a compound or compounds to be tested with an ABIN2 molecule, a TPL-2 molecule and a p105 molecule, under conditions in which, but for the presence of the compound or compounds to be tested, ABIN2, TPL-2 and p105 form a ternary complex with a reference affinity;
determining the binding affinity of the ternary complex of ABIN2, TPL-2 and p105 in the presence of the compound or compounds to be tested; and
selecting those compounds which modulate the binding affinity of the ternary complex with respect to the reference binding affinity.

The method may be simplified, since ABIN2 binds independently to both TPL-2 and p105. Each interaction may be tested for independently. Thus, there is provided a method for identifying a lead compound for a pharmaceutical useful in the treatment of disease, comprising:
incubating a compound or compounds to be tested with an ABIN2 molecule and a TPL-2 molecule, under conditions in which, but for the presence of the compound or compounds to be tested, TPL-2 associates with ABIN2 with a reference affinity;
determining the binding affinity of TPL-2 for ABIN2 in the presence of the compound or compounds to be tested; and
selecting those compounds which modulate the binding affinity of TPL-2 for ABIN2 with respect to the reference binding affinity.

Moreover, the invention provides a method for identifying a lead compound for a pharmaceutical useful in the treatment of disease, comprising: incubating a compound or compounds to be tested with an ABIN2 molecule and a p105 molecule, under conditions in which, but for the presence of the compound or compounds to be tested, p105 associates with ABIN2 with a reference affinity;
determining the binding affinity of p105 for ABIN2 in the presence of the compound or compounds to be tested; and
selecting those compounds which modulate the binding affinity of p105 for ABIN2 with respect to the reference binding affinity.

Preferably, the ABIN2 molecule is a portion of ABIN2 which is responsible for binding to p105 and/or TPL-2, as indicated above. The region encompassed in amino acids 1-250 of ABIN2 comprises all the necessary structure for binding to TPL-2 and p105.

Advantageously, the assay is carried out in vivo in a cell. In a cell based assay, interactions may be measured in a relevant environment. Molecular interactions are detectable, for example, by two-hybrid screens, in which a gene expressing a detectable marker is placed under the control of a promoter which is responsive to a transcription factor assembled by the interaction of the two molecules under test. Other assays may be used to detect molecular interactions, for example co-immunoprecipitation from transfected 293 cell lysates.

Advantageously, the assay comprises the measurement of a biological response. A biological response may be, for example, selected from the group consisting of MEK kinase phosphorylation, MEK kinase activity and ERK kinase activity.

Preferably, the disease is a disease involving or using an inflammatory response.

In a further aspect, the invention relates to a compound identifiable by the method of any one of claims 6 to 15, capable of modulating the direct or indirect interaction of TPL-2 or p105 with ABIN2. For example, such a compound may be an antibody, which is preferably specific for ABIN2. Alternatively, it may be a polypeptide, such as a polypeptide aptamer, or an ABIN2 molecule such as a constitutively active mutant or a dominant negative mutant of ABIN2.

The invention moreover provides a method for modulating the activity of p105 and/or TPL-2 in a cell, comprising administering to the cell a compound as set forth above, as well as a pharmaceutical composition comprising, as active ingredient, a therapeutically effective amount of such and to a method for treating a condition associated with NFκB induction or repression, comprising administering to a subject a therapeutically effective amount of said compound.

In a further aspect, the invention provides a structural model which represents ABIN2 together with TPL-2 and/or p105. TPL-2 has proven to be very difficult to crystallise, in order to obtain a structure which can be used to model TPL-2 in order to assist drug design. Co-crystallisation of TPL-2 and ABIN2, optionally together with p105, allows the structure of TPL-2 to be determined. Advantageously, a co-crystal of the ternary complex is obtained, which is representative of the in vivo complex formed by these molecules.

Preferably, therefore, the model represents a ternary complex comprising ABIN2, p105 and TPL-2 which is derived advantageously from the atomic coordinates of co-crystallised ABIN2, TPL-2 and p105.

This study identifies ABIN-2 as a protein which interacts with both NF-κB1 p105 and TPL-2. In BMDMs, the majority of ABIN-2 and TPL-2 is shown to be associated with p105 in a ternary complex (FIG. 4). ABIN-2 is demonstrated to be essential to maintain steady-state protein levels of TPL-2 but not p105 (FIGS. 7 A and B). Furthermore, NF-κB1 p105/p50 is required to maintain protein levels of both ABIN-2 (FIG. 8) and TPL-2 (2, 31). Thus, in unstimulated cells, ABIN-2 and TPL-2 appear to be confined to a ternary complex with p105 and are not present in isolation.

Binding to p105 dramatically increases the solubility of ABIN-2 (FIG. 2C), suggesting that correct folding of ABIN-2 may require its association with p105. In addition, maintenance of steady-state levels of ABIN-2 protein, but not mRNA, require p105/p50 expression (FIG. 8). While these latter data do not rule out a role for TPL-2 in controlling of ABIN-2 stability (2, 31), they nevertheless demonstrate a close relationship between ABIN-2 and NF-κBI p105/p50. These data also raise the possibility that the phenotype of nfκb1^{-/-} mice (26) may result not only from the lack of p105/p50, but also from deficiency of both TPL-2 (2, 31) and ABIN-2 proteins.

ABIN-2 was originally isolated in a two-hybrid screen using the zinc finger protein A20 as a bait (29). A20 is a primary response gene that is induced by a number of stimuli which activate NF-κB, including TNFα, IL-1 and LPS (4). Analysis of A20-deficient mice has indicated that A20 is required for termination of TNFα-induced NF-κB activation and also for blockade of TNFα-mediated apoptosis (20). Overexpression of ABIN-2 in 293 cells inhibits NF-κB activation by TNFα and it has been suggested that ABIN-2 may contribute to the NF-κB inhibitory function of A20 (29). Surprisingly, however, ABIN-2 knockdown by RNA interference does not affect TNFα or IL-1 activation of an NF-κB reporter gene in 293 cells (FIG. 10). It is therefore unclear whether ABIN-2 acts physiologically to inhibit NF-κB activation.

TPL-2 is the critical MEK kinase required for TLR4 stimulation of the MEK/ERK MAP kinase pathway in LPS-stimulated BMDMs (9). In resting BMDMs, TPL-2 is in a complex with p105, which inhibits TPL-2 MEK kinase activity (31). Consequently, it has been proposed that LPS may activate TPL-2 by promoting its release from p105 (31). The majority of TPL-2 in these cells is also complexed with ABIN-2 (FIG. 4). Together with the observed requirement for ABIN-2 to maintain steady state levels of TPL-2 protein in both HeLa and 293 cells (FIGS. 7 A and B), this strongly suggests that ABIN-2 is involved in the regulation of TPL-2 function. However, ABIN-2 is not associated with the pool of TPL-2 that activates MEK in LPS-stimulated BMDMs (FIG. 9C) and LPS-induced TPL-2 activation correlates with its release from ABIN-2 (FIG. 9D). The importance of TPL-2 release from ABIN-2 is unclear, but ABIN-2 does not appear to function as an inhibitor of TPL-2 MEK kinase activity (FIG. 9E) similar to p105 (2, 31). Together these data raise the possibility that ABIN-2 might function upstream of TPL-2 in the TLR4 signaling pathway that regulates TPL-2 activation.

An alternative possibility is that p105 itself is upstream of and regulates ABIN-2. In this case, ABIN-2 function might be distinct from TPL-2.

ABIN-2 contains a region (residues 298-320), distinct from the TPL-2/p105 binding regions (FIG. 6 B and C), which has homology with regions in both ABIN-1 and the non-catalytic component of the IKK complex, NEMO (13). These homologous regions have been termed ABIN homology domains (AHDs). Interestingly, the AHD of NEMO overlaps with a region required for its oligomerization which is essential for NEMO to couple activating signals from TLR4 to the IKK complex (28). Given this similarity with NEMO, it is possible that ABIN-2 may have an analogous function, perhaps acting as an adapter which couples TPL-2 to signaling pathways downstream of TLR4.

A20 binds to a region of ABIN-2 that is distinct from regions bound by TPL-2 and p105 (FIG. 6). Since LPS triggers upregulation of A20 (4), this raises the possibility that A20 may be recruited to the ABIN-2/TPL-2/p105 complex in LPS-stimulated macrophages. Thus A20 could potentially regulate TLR4 activation of the TPL-2/MEK/ERK MAP kinase signaling pathway in these cells. In an analogous fashion, the NF-κB inhibitory function of A20, which is mediated upstream of IKK (20), is thought to involve its direct recruitment to the IKK complex via NEMO (32).

### BRIEF DESCRIPTION OF THE FIGURES

FIG. 1. ABIN-2 co-purifies with NF-κB1 p105.

(A) Protein was purified from lysate of HeLa S3 cells stably transfected with Ha-p105(S927A) or empty vector (EV) by sequential affinity purification using anti-HA MAb and anti-p105C antibody. Purified protein was resolved by 10%-acrylamide SDS-PAGE and revealed by silver staining. The positions of the identified proteins are shown. For mass spectroscopic analysis, the indicated 100 kDa (bands 1 and 2) and 50 kDa (bands 3-8) regions were excised as a series of adjacent slices numbered from high to low molecular weight from a replicate 10%-acrylamide SDS-PAGE gel stained with colloidal Coomassie brilliant blue (not shown). Proteins in isolated bands were identified by MALDI mass spectroscopy of tryptic digests. (B) ABIN-2 amino acid sequence showing the deduced position of peptides (in bold) identified by MALDI mass spectroscopic analysis of band 7. Peptide coverage corresponded to 56% of the ABIN-2 amino acid sequence. (C) Lysates of HeLa S3 cells were immunoprecipitated with the indicated specific antibodies or pre-immune IgG (PI). Isolated proteins were resolved by 10%-acrylamide SDS-PAGE and Western blotted.

FIG. 2. Transfected ABIN-2-FL co-immunoprecipitates with both Ha-p105 and Myc-TPL-2.

(A) and (B) 293 cells were co-transfected with vectors encoding Ha-p105, Ha-p50, Myc-TPL-2, Ha-p100 or empty vector (EV) and ABIN-2-FL or EV as indicated. Anti-HA, anti-Myc and anti-FL immunoprecipitates and cell lysates were Western blotted with the indicated antibodies. (C) Duplicate cultures of 293 cells were co-transfected with vectors encoding ABIN2-FL and Ha-p105, Myc-TPL-2 or EV. Cell lysates were prepared from each duplicate culture set using either buffer A (1% NP-40) or RIPA buffer, as indicated. Lysates were resolved by 10%-acrylamide SDS-PAGE and Western blotted (upper panels). Ha-p105 and Myc-TPL-2 mRNA levels in total RNA were assayed by semi-quantitative RT-PCR (lower panels). 18SrRNA amplicon was used as an internal control. (D) shows the result of Immunodepletion of endogenouse p105 with anti-p105C antibody.

FIG. 3. ABIN-2 interacts independently to p105 and TPL-2 but preferentially binds to a p105/TPL-2 complex.

(A) 293 cells were co-transfected with vectors encoding Ha-p105 or with no insert (EV) and ABIN-2-FL. Cell lysates were cleared of endogenous TPL-2 by immunoprecipitation with anti-TPL-2 antibody and then re-immunoprecipitated with anti-HA MAb. immunoprecipitates and lysates were Western blotted with the indicated antibodies. (B) 293 cells were co-transfected with the indicated expression vectors. Cell lysates were cleared of endogenous p105 by immunoprecipitation with anti-p105C antibody and then re-immunoprecipitated with anti-FL MAb to isolate ABIN-2-FL and associated Myc-TPL-2. Immunoprecipitates and lysates were Western blotted with the indicated antibodies. (C) 293 cells were co-transfected with vectors encoding Ha-p105 and TPL-2 individually or together. Transfected proteins were affinity purified from cell lysates, prepared in 1% NP-40 buffer A, using GST-ABIN-2 fusion protein coupled to glutathione Sepharose. Isolated proteins were resolved by 10%-acrylamide SDS-PAGE and Western blotted.

FIG. 4. The majority of endogenous ABIN-2 forms a ternary complex with p105 and TPL-2.

(A) BMDM lysate was immunoprecipitated with anti-ABIN-2 antibody or control pre-immune rabbit IgG (PI). Isolated protein was resolved by 10%-acrylamide SDS-PAGE and Western blotted. (B to D) ABIN-2, p105 and TPL-2 were individually removed from lysates of BMDMs by immunoprecipitation with specific antibodies. Control preclearing was carried out using pre-immune rabbit IgG (PI). Precleared lysates were then re-immunoprecipitated with the indicated specific antibodies. Re-immunoprecipitated protein (A and D) and pre-cleared cell lysate (C) was resolved by 10%-acrylamide SDS-PAGE and Western blotted.

FIG. 5. Mapping interacting regions for ABIN-2 on p105 and TPL-2.

(A) Schematic diagram of Ha-p105 mutants. The relative positions of the Rel homology domain (RHD), ankyrin repeats (ANK), death domain (DD) and PEST region are shown. (B) 293 cells were transfected with vectors encoding wild type and mutant forms of Ha-p105. Cell lysates, prepared using 1% Brij-58 buffer A, were incubated with GST-ABIN-2₁₋₄₂₉ fusion protein or GST (control) coupled to glutathione Sepharose beads. Affinity purified protein was resolved by 10%-acrylamide SDS-PAGE and Western blotted. (C) GST-p105₄₉₇₋₉₆₈ fusion protein and GST (control) were coupled to glutathione Sepharose beads and used to affinity purify ABIN-2-FL translated and labeled with [³⁵S]methionine in vitro. Isolated protein was detected by autoradiography of 8%-acrylamide SDS-PAGE. (D) 293 cells were transfected with vectors encoding wild type and mutant forms of Myc-TPL-2. GST-ABIN-2₁₋₄₂₉ was used as an affinity ligand to isolate protein from cell lysates prepared with 1% NP-40 buffer A. Isolated protein was resolved by 10%-acrylamide SDS-PAGE and Western blotted. (E) TPL-2₃₉₈₋₄₆₇ peptide coupled to streptavidin-agarose beads was used as an affinity ligand to isolate ABIN-2-FL from lysates of transfected 293 cells. Bound protein was resolved by 10%-acrylamide SDS-PAGE and Western blotted.

FIG. 6. Mapping regions of ABIN-2 which interact with p105 and TPL-2.

(A) Schematic diagram of recombinant ABIN-2 GST-fusion proteins. The position of the ABIN homology domain (AHD) and the binding regions for TPL-2, p105 and A20 are shown. (B and C) 293 cells were transfected with vectors encoding Myc-p105, Myc-TPL-2 or Myc-A20. Cell lysates were prepared using 1% Brij-58 buffer A and incubated with the indicated GST-ABIN-2 fusion proteins or GST (control) coupled to glutathione Sepharose 4B. Affinity purified protein was resolved by 10%-acrylamide SDS-PAGE and Western blotted.

FIG. 7. Depletion of ABIN-2 by RNA interference dramatically reduces steady-state levels of TPL-2.

(A and B) ABIN-2 expression in HeLa S3 cells (A) and 293 cells (B) was decreased by siRNA treatment (−ABIN-2). Control cells were treated with an irrelevant siRNA oligonucleotide pair (+ABIN-2). Cell lysates were resolved by 10%-acrylamide SDS-PAGE and Western blotted (upper panels). TPL-2 mRNA levels in total RNA were assayed by semi-quantitative RT-PCR (lower panels). 18SrRNA amplicon was used as an internal control. (C) ABIN-2 was depleted by RNA interference in HeLa and 293 cells as in A and B. Expression of p105 and p50 was determined by Western blotting of cell lysates. (D) 293 cells were co-transfected with vectors encoding TPL-2 and ABIN-2-FL or EV. Lysates were resolved by 10%-acrylamide SDS-PAGE and Western blotted (upper panels). In duplicate cultures, transfected TPL-2 mRNA levels were determined by semi-quantitative RT-PCR (lower panels). 18SrRNA was used as an internal control. (E) 293 cells were co-transfected with expression vectors encoding TPL-2 and ABIN-2-FL or EV. After 24 h, cells were metabolically pulse-labeled with [³⁵S]methionine-[³⁵S]cysteine (30 min) and then chased for the times indicated. Anti-TPL-2 immunoprecipitates were resolved by 8%-acrylamide SDS-PAGE and visualized by fluorography. Amounts of immunoprecipitated labelled Myc-TPL-2 were quantified by densitometry (n=3).

FIG. 8. Nf-κb1^{-/-} cells have dramatically reduced levels of ABIN-2 protein.

(A and C) Lysates from nf-κb1^{-/-} and wild type (nf-κb1^{+/+}) 3T3 fibroblasts (A) and BMDMs (C) were immunoprecipitated with the indicated antibodies. Immunoprecipitates and cell lysates were resolved by 10%-acrylamide SDS-PAGE and Western blotted. (B) ABIN-2 mRNA levels in total RNA isolated from nf-κb1^{-/-} and wild type 3T3 fibroblasts were assayed by semi-quantitative PCR. 18SrRNA amplicon was used as an internal control.

FIG. 9. ABIN-2 is not associated with active TPL-2 in LPS-stimulated BMDMs.

(A and B) BMDMs from wild type Balb/C mice were stimulated for the indicated times with LPS or left unstimulated. In B, BMDMs were pre-incubated with MG132 or vehicle control for 30 min prior to stimulation with LPS. Lysates were resolved by 10%-acrylamide SDS-PAGE and Western blotted. (C) BMDMs were stimulated for 15 min with LPS or left unstimulated. Lysates were immunoprecipitated with the indicated antibodies and associated MEK kinase activity determined by coupled MEK/ERK kinase assay. Labelled MBP was visualized by autoradiography after 10%-acrylamide SDS-PAGE. Levels of immunoprecipitated proteins were determined by Western blotting. The LPS-induced mobility shift of M1-TPL-2 was due to phosphorylation (data not shown). (D) BMDM lyates were depleted of ABIN-2 by immunoprecipitation with anti-ABIN-2 antibody. ABIN-2 precleared lysates and control untreated lysates were then Western blotted. (E) 293 cells were co-transfected with vectors encoding Myc-TPL-2 and ABIN-2-FL or EV. The MEK kinase activity of immunoprecipitated Myc-TPL-2 was determined using GST-MEK1 (K207A) protein as a substrate. Phosphorylation was assayed by Western blotting of reaction mixtures and probing with anti-phospho-MEK-1/2 antibody. Western blotting of anti-Myc immunoprecipitates demonstrated similar amounts of TPL-2 were assayed in each reaction.

FIG. 10. ABIN-2 knockdown does not affect TNFα or IL-1 activation of NF-κB.

To determine the role of ABIN-2 in NF-κB activation, ABIN-2 was depleted in 293 cells by RNA interference and NF-κB activation assessed using a luciferase reporter assay. Cells (1×10⁵ cells per well of a 12-well plate; Nunc), precultured for 18 h, were transfected with siRNAs (0.15 nmol/well) using Lipofectamine 2000 (Invitrogen). After 24 h culture, cells were re-transfected with 600 ng of NF-κB luciferase reporter vector (pNF-κB-Luc; Promega) and 20 ng of pTK-Renilla vector (Promega). Cells were recultured for a further 48 h and then stimulated with TNFα (10 g/ml), IL-1 (40 ng/ml; R and D Systems) or with no addition for 16 h and lysed with 200 μl of passive lysis buffer (Promega). (A) Luciferase and renilla activities in cell lysates were determined using the dual-assay system (Promega) and NF-κB activity deduced by normalizing luciferase values against the Renilla transfection efficiency control. Mean data are presented from replicate cultures (n=3), normalized against Renilla. (B) Knockdown of ABIN-2 was confirmed by Western blotting of cell lysates.

### DETAILED DESCRIPTION OF THE INVENTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### 1. ABIN2

ABIN2 was initially cloned in a yeast two-hybrid assay where A20 was used as a bait in the screening of a murine fibrosarcoma L929r2 cDNA library (27; the disclosure of which is incorporated herein by reference). ABIN2 was initially proposed to be an inhibitor of NFκB.

ABIN2 also interacts with the endothelial receptor Tie2. This receptor is essential for blood vessel formation and promotes endothelial survival. In a further yeast two-hybrid screening of a human endothelial cell cDNA library, ABIN2 was identified as interacting with the intracellular domain of the Tie2 receptor. Coexpression of Tie2 and ABIN2 in CHO cells confirmed the interaction occurs in mammalian cells. Deletion analysis identified the Tie2 binding motif to be encompassed between residues 171 and 272 in ABIN2. The complete sequence of ABIN2 is available from GenBank under accession number CAC34835 [gi: 13445188].

We have now determined ABIN2 to be a p105-associated protein, which binds both p105 and TPL-2.

1a. The ABIN2 Molecule

As used herein, “an ABIN2 molecule” refers to a polypeptide having at least one biological activity of ABIN2. The term thus includes fragments of ABIN2 which retain at least one structural determinant and/or functional or binding activity of ABIN2.

The preferred ABIN2 molecule has the structure set forth in GenBank (accession No. CAC34835 [gi: 13445188]). This polypeptide, human ABIN2, is encoded by the nucleic acid sequence set forth under accession no: AJ304866 [gi:13445187]. Alternative sequences encoding the polypeptide of CAC34835 may be designed, having regard to the degeneracy of the genetic code, by persons skilled in the art. Moreover, the invention includes ABIN2 polypeptides which are encoded by sequences which have substantial homology to the nucleic acid sequence set forth in CAC34835. “Substantial homology”, where homology indicates sequence identity, means more than 40% sequence identity, preferably more than 45% sequence identity and most preferably a sequence identity of 50%, 60%, 70%, 80%, 90% or more, as judged by direct sequence alignment and comparison.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

### Variants and Derivatives

The terms “variant” or “derivative” in relation to the amino acid sequences as described here includes any substitution of, variation of, modification of, replacement of, deletion of or addition of one (or more) amino acids from or to the sequence. Preferably, the resultant amino acid sequence retains substantially the same activity as the unmodified sequence, preferably having at least the same activity as the full length polypeptides described herein. Thus, the key feature of the sequences—namely that they are capable of forming a stable ternary complex—is preferably retained.

Polypeptides having the amino acid sequence shown in the Examples, or fragments or homologues thereof may be modified for use in the methods and compositions described here. Typically, modifications are made that maintain the biological activity of the sequence. Amino acid substitutions may be made, for example from 1, 2 or 3 to 10, 20 or 30 substitutions provided that the modified sequence retains the biological activity of the unmodified sequence. Amino acid substitutions may include the use of non-naturally occurring analogues, for example to increase blood plasma half-life of a therapeutically administered polypeptide.

Natural variants of ABIN2 and other polypeptides derived herein are likely to comprise conservative amino acid substitutions. Conservative substitutions may be defined, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The invention moreover encompasses polypeptides encoded by nucleic acid sequences capable of hybridising to the nucleic acid sequence set forth in GenBank AJ304866 at any one of low, medium or high stringency.

Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency refers to conditions that permit hybridisation of only those nucleic acid sequences that form stable hybrids in 1 M Na+ at 65-68° C. High stringency conditions can be provided, for example, by hybridisation in an aqueous solution containing 6×SSC, 5×Denhardt's, 1% SDS (sodium dodecyl sulphate), 0.1 Na⁺ pyrophosphate and 0.1 mg/ml denatured salmon sperm DNA as non specific competitor. Following hybridisation, high stringency washing may be done in several steps, with a final wash (about 30 min) at the hybridisation temperature in 0.2-0.1×SSC, 0.1% SDS.

Moderate stringency refers to conditions equivalent to hybridisation in the above described solution but at about 60-62° C. In that case the final wash is performed at the hybridisation temperature in 1×SSC, 0.1% SDS.

Low stringency refers to conditions equivalent to hybridisation in the above described solution at about 50-52° C. In that case, the final wash is performed at the hybridisation temperature in 2×SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the probe also play a role.

Advantageously, the invention moreover provides nucleic acid sequence which are capable of hybridising, under stringent conditions, to a fragment of the nucleic acid sequence set forth in GenBank AJ304866. Preferably, the fragment is between 15 and 50 bases in length. Advantageously, it is about 25 bases in length.

Given the guidance provided herein, the nucleic acids of the invention are obtainable according to methods well known in the art. For example, a DNA of the invention is obtainable by chemical synthesis, using polymerase chain reaction (PCR) or by screening a genomic library or a suitable cDNA library prepared from a source believed to possess ABIN2 and to express it at a detectable level.

Chemical methods for synthesis of a nucleic acid of interest are known in the art and include triester, phosphite, phosphoramidite and H-phosphonate methods, PCR and other autoprimer methods as well as oligonucleotide synthesis on solid supports. These methods may be used if the entire nucleic acid sequence of the nucleic acid is known, or the sequence of the nucleic acid complementary to the coding strand is available. Alternatively, if the target amino acid sequence is known, one may infer potential nucleic acid sequences using known and preferred coding residues for each amino acid residue.

An alternative means to isolate the gene encoding ABIN2 is to use PCR technology as described e.g. in section 14 of Sambrook et al., 1989. This method requires the use of oligonucleotide probes that will hybridise to ABIN2 nucleic acid. Strategies for selection of oligonucleotides are described below.

Libraries are screened with probes or analytical tools designed to identify the gene of interest or the protein encoded by it. For cDNA expression libraries suitable means include monoclonal or polyclonal antibodies that recognise and specifically bind to ABIN2; oligonucleotides of about 20 to 80 bases in length that encode known or suspected ABIN2 cDNA from the same or different species; and/or complementary or homologous cDNAs or fragments thereof that encode the same or a hybridising gene. Appropriate probes for screening genomic DNA libraries include, but are not limited to oligonucleotides, cDNAs or fragments thereof that encode the same or hybridising DNA; and/or homologous genomic DNAs or fragments thereof.

A nucleic acid encoding ABIN2 may be isolated by screening suitable cDNA or genomic libraries under suitable hybridisation conditions with a probe, i.e. a nucleic acid disclosed herein including oligonucleotides derivable from the sequences set forth in GenBank accession no. CAC34835. Suitable libraries are commercially available or can be prepared e.g. from cell lines, tissue samples, and the like.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases set forth in AJ304866. The nucleic acid sequences selected as probes should be of sufficient length and sufficiently unambiguous so that false positive results are minimised. The nucleotide sequences are usually based on conserved or highly homologous nucleotide sequences or regions of ABIN2. The nucleic acids used as probes may be degenerate at one or more positions. The use of degenerate oligonucleotides may be of particular importance where a library is screened from a species in which preferential codon usage in that species is not known.

Preferred regions from which to construct probes include 5′ and/or 3′ coding sequences, sequences predicted to encode ligand binding sites, and the like. For example, either the full-length cDNA clone disclosed herein or fragments thereof can be used as probes. Preferably, nucleic acid probes of the invention are labelled with suitable label means for ready detection upon hybridisation. For example, a suitable label means is a radiolabel. The preferred method of labelling a DNA fragment is by incorporating α³²P dATP with the Klenow fragment of DNA polymerase in a random priming reaction, as is well known in the art. Oligonucleotides are usually end-labelled with γ³²P-labelled ATP and polynucleotide kinase. However, other methods (e.g. non-radioactive) may also be used to label the fragment or oligonucleotide, including e.g. enzyme labelling, fluorescent labelling with suitable fluorophores and biotinylation.

After screening the library, e.g. with a portion of DNA including substantially the entire ABIN2-encoding sequence or a suitable oligonucleotide based on a portion of said DNA, positive clones are identified by detecting a hybridisation signal; the identified clones are characterised by restriction enzyme mapping and/or DNA sequence analysis, and then examined, e.g. by comparison with the sequences set forth herein, to ascertain whether they include DNA encoding a complete ABIN2 (i.e., if they include translation initiation and termination codons). If the selected clones are incomplete, they may be used to rescreen the same or a different library to obtain overlapping clones. If the library is genomic, then the overlapping clones may include exons and introns. If the library is a cDNA library, then the overlapping clones will include an open reading frame. In both instances, complete clones may be identified by comparison with the DNAs and deduced amino acid sequences provided herein.

“Structural determinant” means that the derivative in question retains at least one structural feature of ABIN2. Structural features include possession of a structural motif that is capable of replicating at least one biological activity of naturally occurring ABIN2 polypeptide. Thus ABIN2 as provided by the present invention includes splice variants encoded by mRNA generated by alternative splicing of a primary transcript, amino acid mutants, glycosylation variants and other covalent derivatives of ABIN2 which retain at least one physiological and/or physical property of ABIN2. Exemplary derivatives include molecules wherein the protein of the invention is covalently modified by substitution, chemical, enzymatic, or other appropriate means with a moiety other than a naturally occurring amino acid. Such a moiety may be a detectable moiety such as an enzyme or a radioisotope. Further included are naturally occurring variants of ABIN2 found with a particular species, preferably a mammal. Such a variant may be encoded by a related gene of the same gene family, by an allelic variant of a particular gene, or represent an alternative splicing variant of the ABIN2 gene.

It has been observed that the N-terminal 1-250 amino acids of ABIN2 are necessary for interaction with p105. Thus, the ABIN2 molecule according to the invention preferably retains the C-terminal portion of naturally occurring ABIN2. Preferably, the ABIN2 molecule according to the present invention retains at least amino acids 1-250 of naturally occurring ABIN2, for example ABIN2 as represented in AJ304866.

Advantageously, the ABIN2 molecule according to the invention comprises amino acids 90-250 of ABIN2; preferably amino acids 130-250 of ABIN2; and most preferably amino acids 194-250 of ABIN2. The latter truncations are capable of binding TPL-2, but not p105.

Moreover, the invention extends to homologues of such fragments as defined above.

Derivatives which retain common structural determinants can, as indicated above, be fragments of ABIN2. Fragments of ABIN2 comprise individual domains thereof, as well as smaller polypeptides derived from the domains. Preferably, smaller polypeptides derived from ABIN2 according to the invention define a single functional domain which is characteristic of ABIN2. Fragments may in theory be almost any size, as long as they retain one characteristic of ABIN2. Preferably, fragments will be between 4 and 300 amino acids in length. Longer fragments are regarded as truncations of the full-length ABIN2 and generally encompassed by the term “ABIN2”.

Derivatives of ABIN2 also comprise mutants thereof, which may contain amino acid deletions, additions or substitutions, subject to the requirement to maintain at least one feature characteristic of ABIN2. Thus, conservative amino acid substitutions may be made substantially without altering the nature of ABIN2, as may truncations from the N terminus. Deletions and substitutions may moreover be made to the fragments of ABIN2 comprised by the invention. ABIN2 mutants may be produced from a DNA encoding ABIN2 which has been subjected to in vitro mutagenesis resulting e.g. in an addition, exchange and/or deletion of one or more amino acids. For example, substitutional, deletional or insertional variants of ABIN2 can be prepared by recombinant methods and screened for immuno-crossreactivity with the native forms of ABIN2.

The fragments, mutants and other derivatives of ABIN2 preferably retain substantial homology with ABIN2. As used herein, “homology” means that the two entities share sufficient characteristics for the skilled person to determine that they are similar in origin and function. Preferably, homology is used to refer to sequence identity, and is determined as defined above.

2c. Preparation of an ABIN2 Molecule

The invention encompasses the production of ABIN2 molecules for use inter alia in the stabilisation of TPL-2 as described above. Preferably, ABIN2 molecules are produced by recombinant DNA technology, by means of which a nucleic acid encoding a ABIN2 molecule can be incorporated into a vector for further manipulation. As used herein, vector (or plasmid) refers to discrete elements that are used to introduce heterologous DNA into cells for either expression or replication thereof. Selection and use of such vehicles are well within the skill of the artisan. Many vectors are available, and selection of appropriate vector will depend on the intended use of the vector, i.e. whether it is to be used for DNA amplification or for DNA expression, the size of the DNA to be inserted into the vector, and the host cell to be transformed with the vector. Each vector contains various components depending on its function (amplification of DNA or expression of DNA) and the host cell for which it is compatible. The vector components generally include, but are not limited to, one or more of the following: an origin of replication, one or more marker genes, an enhancer element, a promoter, a transcription termination sequence and a signal sequence.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR322 is suitable for most Gram-negative bacteria, the 2μ plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Most expression vectors are shuttle vectors, i.e. they are capable of replication in at least one class of organisms but can be transfected into another class of organisms for expression. For example, a vector is cloned in E. coli and then the same vector is transfected into yeast or mammalian cells even though it is not capable of replicating independently of the host cell chromosome. DNA may also be replicated by insertion into the host genome. However, the recovery of genomic DNA encoding ABIN2 is more complex than that of exogenously replicated vector because restriction enzyme digestion is required to excise ABIN2 DNA. DNA can be amplified by PCR and be directly transfected into the host cells without any replication component.

Advantageously, an expression and cloning vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

As to a selective gene marker appropriate for yeast, any marker gene can be used which facilitates the selection for transformants due to the phenotypic expression of the marker gene.

Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

Since the replication of vectors is conveniently done in E. coli, an E. coli genetic marker and an E. coli origin of replication are advantageously included. These can be obtained from E. coli plasmids, such as pBR322, Bluescript® vector or a pUC plasmid, e.g. pUC18 or pUC19, which contain both E. coli replication origin and E. coli genetic marker conferring resistance to antibiotics, such as ampicillin.

Suitable selectable markers for mammalian cells are those that enable the identification of cells competent to take up ABIN2 nucleic acid, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked DNA that encodes ABIN2. Amplification is the process by which genes in greater demand for the production of a protein critical for growth, together with closely associated genes which may encode a desired protein, are reiterated in tandem within the chromosomes of recombinant cells. Increased quantities of desired protein are usually synthesised from thus amplified DNA.

Expression and cloning vectors usually contain a promoter that is recognised by the host organism and is operably linked to ABIN2 nucleic acid. Such a promoter may be inducible or constitutive. The promoters are operably linked to DNA encoding ABIN2 by removing the promoter from the source DNA by restriction enzyme digestion and inserting the isolated promoter sequence into the vector. Both the native ABIN2 promoter sequence and many heterologous promoters may be used to direct amplification and/or expression of ABIN2 DNA. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Their nucleotide sequences have been published, thereby enabling the skilled worker operably to ligate them to DNA encoding ABIN2, using linkers or adaptors to supply any required restriction sites. Promoters for use in bacterial systems will also generally contain a Shine-Delgarno sequence operably linked to the DNA encoding ABIN2.

Preferred expression vectors are bacterial expression vectors which comprise a promoter of a bacteriophage such as phagex or T7 which is capable of functioning in the bacteria. In one of the most widely used expression systems, the nucleic acid encoding the fusion protein may be transcribed from the vector by T7 RNA polymerase (Studier et al, Methods in Enzymol. 185; 60-89, 1990). In the E. coli BL21(DE3) host strain, used in conjunction with pET vectors, the T7 RNA polymerase is produced from the λ-lysogen DE3 in the host bacterium, and its expression is under the control of the IPTG inducible lac UV5 promoter. This system has been employed successfully for over-production of many proteins. Alternatively the polymerase gene may be introduced on a lambda phage by infection with an int-phage such as the CE6 phage which is commercially available (Novagen, Madison, USA). other vectors include vectors containing the lambda PL promoter such as PLEX (Invitrogen, NL), vectors containing the trc promoters such as pTrcHisXpressTm (Invitrogen) or pTrc99 (Pharmacia Biotech, SE), or vectors containing the tac promoter such as pKK223-3 (Pharmacia Biotech) or PMAL (new England Biolabs, MA, USA).

Moreover, the ABIN2 gene according to the invention preferably includes a secretion sequence in order to facilitate secretion of the polypeptide from bacterial hosts, such that it will be produced as a soluble native peptide rather than in an inclusion body. The peptide may be recovered from the bacterial periplasmic space, or the culture medium, as appropriate.

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the a- or α-factor or a promoter derived from a gene encoding a glycolytic enzyme such as the promoter of the enolase, glyceraldehyde-3-phosphate dehydrogenase (GAP), 3-phospho glycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, the S. cerevisiae GAL 4 gene, the S. pombe nmt 1 gene or a promoter from the TATA binding protein (TBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PH05 promoter is e.g. a shortened acid phosphatase PH05 promoter devoid of the upstream regulatory elements (UAS) such as the PH05 (−173) promoter element starting at nucleotide −173 and ending at nucleotide −9 of the PH05 gene.

ABIN2 gene transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g. a ribosomal protein promoter, and from the promoter normally associated with ABIN2 sequence, provided such promoters are compatible with the host cell systems.

Transcription of a DNA encoding ABIN2 by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to ABIN2 DNA, but is preferably located at a site 5′ from the promoter.

Advantageously, a eukaryotic expression vector encoding ABIN2 may comprise a locus control region (LCR). LCRs are capable of directing high-level integration site independent expression of transgenes integrated into host cell chromatin, which is of importance especially where the ABIN2 gene is to be expressed in the context of a permanently-transfected eukaryotic cell line in which chromosomal integration of the vector has occurred, in vectors designed for gene therapy applications or in transgenic animals.

Eukaryotic expression vectors will also contain sequences necessary for the termination of transcription and for stabilising the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding ABIN2.

An expression vector includes any vector capable of expressing ABIN2 nucleic acids that are operatively linked with regulatory sequences, such as promoter regions, that are capable of expression of such DNAs. Thus, an expression vector refers to a recombinant DNA or RNA construct, such as a plasmid, a phage, recombinant virus or other vector, that upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, DNAs encoding ABIN2 may be inserted into a vector suitable for expression of cDNAs in mammalian cells, e.g. a CMV enhancer-based vector such as pEVRF (Matthias, et al., (1989) NAR 17, 6418).

Particularly useful for practicing the present invention are expression vectors that provide for the transient expression of DNA encoding ABIN2 in mammalian cells. Transient expression usually involves the use of an expression vector that is able to replicate efficiently in a host cell, such that the host cell accumulates many copies of the expression vector, and, in turn, synthesises high levels of ABIN2. For the purposes of the present invention, transient expression systems are useful e.g. for identifying ABIN2 mutants, to identify potential phosphorylation sites, or to characterise functional domains of the protein.

Construction of vectors according to the invention employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing ABIN2 expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe which may be based on a sequence provided herein. Those skilled in the art will readily envisage how these methods may be modified, if desired.

Thus, the invention comprises host cells transformed with vectors encoding a heterologous ABIN2 molecule. As used herein, a heterologous ABIN2 molecule may be a mutated form of the endogenous ABIN2, or a mutated or wild-type form of an exogenous ABIN2.

ABIN2 may advantageously be expressed in insect cell systems. Insect cells suitable for use in the method of the invention include, in principle, any lepidopteran cell which is capable of being transformed with an expression vector and expressing heterologous proteins encoded thereby. In particular, use of the Sf cell lines, such as the Spodoptera frugiperda cell line IPBL-SF-21 AE (Vaughn et al., (1977) In Vitro, 13, 213-217) is preferred. The derivative cell line Sf9 is particularly preferred. However, other cell lines, such as Tricoplusia ni 368 (Kurstack and Marmorosch, (1976) Invertebrate Tissue Culture Applications in Medicine, Biology and Agriculture. Academic Press, New York, USA) may be employed. These cell lines, as well as other insect cell lines suitable for use in the invention, are commercially available (e.g. from Stratagene, La Jolla, Calif., USA).

As well as expression in insect cells in culture, the invention also comprises the expression of ABIN2 proteins in whole insect organisms. The use of virus vectors such as baculovirus allows infection of entire insects, which are in some ways easier to grow than cultured cells as they have fewer requirements for special growth conditions. Large insects, such as silk moths, provide a high yield of heterologous protein. The protein can be extracted from the insects according to conventional extraction techniques.

Expression vectors suitable for use in the invention include all vectors which are capable of expressing foreign proteins in insect cell lines. In general, vectors which are useful in mammalian and other eukaryotic cells are also applicable to insect cell culture. Baculovirus vectors, specifically intended for insect cell culture, are especially preferred and are widely obtainable commercially (e.g. from Invitrogen and Clontech). Other virus vectors capable of infecting insect cells are known, such as Sindbis virus (Hahn et al., (1992) PNAS (USA) 89, 2679-2683). The baculovirus vector of choice (reviewed by Miller (1988) Ann. Rev. Microbiol. 42, 177-199) is Autographa californica multiple nuclear polyhedrosis virus, AcMNPV.

Typically, the heterologous gene replaces at least in part the polyhedrin gene of AcMNPV, since polyhedrin is not required for virus production. In order to insert the heterologous gene, a transfer vector is advantageously used. Transfer vectors are prepared in E. coli hosts and the DNA insert is then transferred to AcMNPV by a process of homologous recombination.

### 2. ABIN2 is a TPL-2 Stabiliser

In one aspect, the invention relates to the use of an ABIN2 molecule for the stabilisation of TPL-2 in vitro and in vivo, as well as for the modulation of TPL2 activity.

2a. Uses of the ABIN2 Molecule

The invention includes, for example, the use of ABIN2 molecules to modulate TPL-2 activity in in vitro and/or in vivo assays, and in particular to stabilise TPL-2 in such assay systems; the use of an ABIN2 molecule to modulate TPL-2 activity in a cell in vivo, for example in order to induce or prevent an immune reaction or an inflammatory response. In an advantageous embodiment, the invention relates to the use of an ABIN2 molecule in the treatment of a disease associated with inflammation.

ABIN2 is able to stabilise TPL-2, especially in the context of a ternary complex of ABIN2 and p105. Advantageously, therefore, ABIN2 is useful in in vivo and in vitro assays involving TPL-2 and/or p105, to mimic the conditions prevalent in vivo in which TPL-2 is stabilised by ABIN2.

ABIN2 is also useful in the preparation of molecular models which can be used to represent TPL-2 in drug design assays. Co-crystals of TPL-2, ABIN2 and p105 display enhanced stability and allow the determination of a crystal structure, which can be used to generate such a molecular model.

2b. The TPL-2 Molecule

As used herein, “a TPL-2 molecule” refers to a polypeptide having at least one biological activity of TPL-2. The term thus includes fragments of TPL-2 which retain at least one structural determinant of TPL-2.

The preferred TPL-2 molecule has the structure set forth in GenBank (accession No. M94454). This polypeptide, rat TPL-2, is encoded by the nucleic acid sequence also set forth under accession no M94454. Alternative sequences encoding the polypeptide of M94454 may be designed, having regard to the degeneracy of the genetic code, by persons skilled in the art. Moreover, the invention includes TPL-2 polypeptides which are encoded by sequences which have substantial homology to the nucleic acid sequence set forth in M94454. “Substantial homology”, where homology indicates sequence identity, means more than 40% sequence identity, preferably more than 45% sequence identity and most preferably a sequence identity of 50% or more, as judged by direct sequence alignment and comparison.

For example, the term “a TPL-2 molecule” refers to COT, the human homologue of TPL-2. COT is 90% identical to TPL-2.

Sequence homology (or identity) may be determined as set out above for ABIN2.

The invention moreover encompasses polypeptides encoded by nucleic acid sequences capable of hybridising to the nucleic acid sequence set forth in GenBank M94454 at any one of low, medium or high stringency.

Stringency of hybridisation refers to conditions under which polynucleic acids hybrids are stable, as set forth above for ABIN2.

The invention also refers to homologues, variants, derivatives, fragments and other embodiments of the TPL-2 molecule, which are defined as for ABIN2, above.

### 3. ABIN2 is a Drug Development Target

According to the present invention, an ABIN2 molecule is used as a target to identify compounds, for example lead compounds for pharmaceuticals, which are capable of modulating the activity of TPL-2 and/or p105 in the MEK/ERK kinase pathway. Accordingly, the invention relates to an assay and provides a method for identifying a compound or compounds capable, directly or indirectly, of modulating the activity of TPL-2 and/or p105, comprising the steps of:

(a) incubating an ABIN2 molecule with the compound or compounds to be assessed; and

(b) identifying those compounds which influence the activity of the ABIN2 molecule.

3a. ABIN2 Binding Compounds

According to a first embodiment of this aspect invention, the assay is configured to detect polypeptides which bind directly to the ABIN2 molecule.

The invention therefore provides a method for identifying a modulator of TPL2 and/or p105 activity, comprising the steps of:
(a) incubating a ABIN2 molecule with the compound or compounds to be assessed; and
(b) identifying those compounds which bind to the ABIN2 molecule.

Preferably, the method further comprises the step of:
(c) assessing the compounds which bind to ABIN2 for the ability to modulate TPL-2 and/or p105 activation in a cell-based assay.

Binding to ABIN2 may be assessed by any technique known to those skilled in the art. Examples of suitable assays include the two hybrid assay system, which measures interactions in vivo, affinity chromatography assays, for example involving binding to polypeptides immobilised on a column, fluorescence assays in which binding of the compound(s) and TPL-2 is associated with a change in fluorescence of one or both partners in a binding pair, and the like. Preferred are assays performed in vivo in cells, such as the two-hybrid assay.

In a preferred aspect of this embodiment, the invention provides a method for identifying a lead compound for a pharmaceutical useful in the treatment of disease involving or using an inflammatory response, comprising incubating a compound or compounds to be tested with an ABIN2 molecule and TPL-2 and/or p105, under conditions in which, but for the presence of the compound or compounds to be tested, ABIN2 associates with TPL-2 and/or p105 with a reference affinity;
determining the binding affinity of ABIN2 for TPL-2 and/orp105 in the presence of the compound or compounds to be tested; and
selecting those compounds which modulate the binding affinity of ABIN2 for TPL-2 and/or p105 with respect to the reference binding affinity.

Preferably, therefore, the assay according to the invention is calibrated in absence of the compound or compounds to be tested, or in the presence of a reference compound whose activity in binding to ABIN2 is known or is otherwise desirable as a reference value. For example, in a two-hybrid system, a reference value may be obtained in the absence of any compound. Addition of a compound or compounds which increase the binding affinity of ABIN2 for TPL-2 and/or p105 increases the readout from the assay above the reference level, whilst addition of a compound or compounds which decrease this affinity results in a decrease of the assay readout below the reference level.

### 4. Compounds

In a still further aspect, the invention relates to a compound or compounds identifiable by an assay method as defined in the previous aspect of the invention.

Compounds which influence the TPL-2/ABIN2/p105 interaction may be of almost any general description, including low molecular weight compounds, including organic compounds which may be linear, cyclic, polycyclic or a combination thereof, peptides, polypeptides including antibodies, or proteins. In general, as used herein, “peptides”, “polypeptides” and “proteins” are considered equivalent.

3a. Antibodies

Antibodies, as used herein, refers to complete antibodies or antibody fragments capable of binding to a selected target, and including Fv, ScFv, Fab′ and F(ab′)₂, monoclonal and polyclonal antibodies, engineered antibodies including chimeric, CDR-grafted and humanised antibodies, and artificially selected antibodies produced using phage display or alternative techniques. Small fragments, such Fv and ScFv, possess advantageous properties for diagnostic and therapeutic applications on account of their small size and consequent superior tissue distribution.

The antibodies according to the invention are especially indicated for diagnostic and therapeutic applications. Accordingly, they may be altered antibodies comprising an effector protein such as a toxin or a label. Especially preferred are labels which allow the imaging of the distribution of the antibody in vivo. Such labels may be radioactive labels or radioopaque labels, such as metal particles, which are readily visualisable within the body of a patient. Moreover, the may be fluorescent labels or other labels which are visualisable on tissue samples removed from patients.

Recombinant DNA technology may be used to improve the antibodies of the invention. Thus, chimeric antibodies may be constructed in order to decrease the immunogenicity thereof in diagnostic or therapeutic applications. Moreover, immunogenicity may be minimised by humanising the antibodies by CDR grafting [see European Patent Application 0 239 400 (Winter)] and, optionally, framework modification [see EP0 239 400; reviewed in international patent application WO 90/07861 (Protein Design Labs)].

Antibodies according to the invention may be obtained from animal serum, or, in the case of monoclonal antibodies or fragments thereof, produced in cell culture. Recombinant DNA technology may be used to produce the antibodies according to established procedure, in bacterial or preferably mammalian cell culture. The selected cell culture system preferably secretes the antibody product.

Therefore, the present invention includes a process for the production of an antibody according to the invention comprising culturing a host, e.g. E. coli or a mammalian cell, which has been transformed with a hybrid vector comprising an expression cassette comprising a promoter operably linked to a first DNA sequence encoding a signal peptide linked in the proper reading frame to a second DNA sequence encoding said protein, and isolating said protein.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. foetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production provides relatively pure antibody preparations and allows scale-up to give large amounts of the desired antibodies. Techniques for bacterial cell, yeast or mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436597, which are incorporated herein by reference.

The cell culture supernatants are screened for the desired antibodies, preferentially by immunofluorescent staining of cells expressing ABIN2 by immunoblotting, by an enzyme immunoassay, e.g. a sandwich assay or a dot-assay, or a radioimmunoassay.

For isolation of the antibodies, the immunoglobulins in the culture supernatants or in the ascitic fluid may be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-) affinity chromatography, e.g. affinity chromatography with an ABIN2 molecule or with Protein-A.

The invention further concerns hybridoma cells secreting the monoclonal antibodies of the invention. The preferred hybridoma cells of the invention are genetically stable, secrete monoclonal antibodies of the invention of the desired specificity and can be activated from deep-frozen cultures by thawing and recloning.

The invention also concerns a process for the preparation of a hybridoma cell line secreting monoclonal antibodies directed to an ABIN2 molecule, characterised in that a suitable mammal, for example a Balb/c mouse, is immunised with a purified ABIN2 molecule, an antigenic carrier containing a purified ABIN2 molecule or with cells bearing ABIN2, antibody-producing cells of the immunised mammal are fused with cells of a suitable myeloma cell line, the hybrid cells obtained in the fusion are cloned, and cell clones secreting the desired antibodies are selected. For example spleen cells of Balb/c mice immunised with cells bearing TPL-2 are fused with cells of the myeloma cell line PAI or the myeloma cell line Sp2/0-Ag14, the obtained hybrid cells are screened for secretion of the desired antibodies, and positive hybridoma cells are cloned.

Preferred is a process for the preparation of a hybridoma cell line, characterised in that Balb/c mice are immunised by injecting subcutaneously and/or intraperitoneally between 10 and 107 and 108 cells of human tumour origin which express ABIN2 containing a suitable adjuvant several times, e.g. four to six times, over several months, e.g. between two and four months, and spleen cells from the immunised mice are taken two to four days after the last injection and fused with cells of the myeloma cell line PAI in the presence of a fusion promoter, preferably polyethylene glycol. Preferably the myeloma cells are fused with a three- to twentyfold excess of spleen cells from the immunised mice in a solution containing about 30% to about 50% polyethylene glycol of a molecular weight around 4000. After the fusion the cells are expanded in suitable culture media as described hereinbefore, supplemented with a selection medium, for example HAT medium, at regular intervals in order to prevent normal myeloma cells from overgrowing the desired hybridoma cells.

The invention also concerns recombinant DNAs comprising an insert coding for a heavy chain variable domain and/or for a light chain variable domain of antibodies directed to an ABIN2 molecule as described hereinbefore. By definition such DNAs comprise coding single stranded DNAs, double stranded DNAs consisting of said coding DNAs and of complementary DNAs thereto, or these complementary (single stranded) DNAs themselves.

Furthermore, DNA encoding a heavy chain variable domain and/or for a light chain variable domain of antibodies directed to an ABIN2 molecule can be enzymatically or chemically synthesised DNA having the authentic DNA sequence coding for a heavy chain variable domain and/or for the light chain variable domain, or a mutant thereof. A mutant of the authentic DNA is a DNA encoding a heavy chain variable domain and/or a light chain variable domain of the above-mentioned antibodies in which one or more amino acids are deleted or exchanged with one or more other amino acids. Preferably said modification(s) are outside the CDRs of the heavy chain variable domain and/or of the light chain variable domain of the antibody. Such a mutant DNA is also intended to be a silent mutant wherein one or more nucleotides are replaced by other nucleotides with the new codons coding for the same amino acid(s). Such a mutant sequence is also a degenerated sequence. Degenerated sequences are degenerated within the meaning of the genetic code in that an unlimited number of nucleotides are replaced by other nucleotides without resulting in a change of the amino acid sequence originally encoded. Such degenerated sequences may be useful due to their different restriction sites and/or frequency of particular codons which are preferred by the specific host, particularly E. coli, to obtain an optimal expression of the heavy chain murine variable domain and/or a light chain murine variable domain.

The term mutant is intended to include a DNA mutant obtained by in vitro mutagenesis of the authentic DNA according to methods known in the art.

For the assembly of complete tetrameric immunoglobulin molecules and the expression of chimeric antibodies, the recombinant DNA inserts coding for heavy and light chain variable domains are fused with the corresponding DNAs coding for heavy and light chain constant domains, then transferred into appropriate host cells, for example after incorporation into hybrid vectors.

The invention therefore also concerns recombinant DNAs comprising an insert coding for a heavy chain murine variable domain of an antibody directed TPL-2 fused to a human constant domain g, for example γ1, γ2, γ3 or γ4, preferably γ1 or γ4. Likewise the invention concerns recombinant DNAs comprising an insert coding for a light chain murine variable domain of an antibody directed to ABIN2 fused to a human constant domain κ or λ, preferably κ.

In another embodiment the invention pertains to recombinant DNAs coding for a recombinant polypeptide wherein the heavy chain variable domain and the light chain variable domain are linked by way of a spacer group, optionally comprising a signal sequence facilitating the processing of the antibody in the host cell and/or a DNA coding for a peptide facilitating the purification of the antibody and/or a cleavage site and/or a peptide spacer and/or an effector molecule.

The DNA coding for an effector molecule is intended to be a DNA coding for the effector molecules useful in diagnostic or therapeutic applications. Thus, effector molecules which are toxins or enzymes, especially enzymes capable of catalysing the activation of prodrugs, are particularly indicated. The DNA encoding such an effector molecule has the sequence of a naturally occurring enzyme or toxin encoding DNA, or a mutant thereof, and can be prepared by methods well known in the art.

Antibodies and antibody fragments according to the invention are useful in diagnosis and therapy. Accordingly, the invention provides a composition for therapy or diagnosis comprising an antibody according to the invention.

In the case of a diagnostic composition, the antibody is preferably provided together with means for detecting the antibody, which may be enzymatic, fluorescent, radioisotopic or other means. The antibody and the detection means may be provided for simultaneous, simultaneous separate or sequential use, in a diagnostic kit intended for diagnosis.

4b. Peptides

Peptides according to the present invention are usefully derived from ABIN2, TPL-2, p105 or another polypeptide involved in the functional ABIN2/TPL-2/p105 interaction. Preferably, the peptides are derived from the domains in ABIN2, TPL-2 or p105 which are responsible for p105/TPL-2/ABIN2 interaction. For example, Thomberry et al., (1994) Biochemistry 33:3934-3940 and Milligan et al., (1995) Neuron 15:385-393 describe the use of modified tetrapeptides to inhibit ICE protease. In an analogous fashion, peptides derived from ABIN2, TPL-2, p105 or an interacting protein may be modified, for example with an aldehyde group, chloromethylketone, (acyloxy) methyl ketone or CH₂OC(O)-DCB group to inhibit the ABIN2/TPL-2/p105 interaction.

In order to facilitate delivery of peptide compounds to cells, peptides may be modified in order to improve their ability to cross a cell membrane. For example, U.S. Pat. No. 5,149,782 discloses the use of fusogenic peptides, ion-channel forming peptides, membrane peptides, long-chain fatty acids and other membrane blending agents to increase protein transport across the cell membrane. These and other methods are also described in WO 97/37016 and U.S. Pat. No. 5,108,921, incorporated herein by reference.

Many compounds according to the present invention may be lead compounds useful for drug development. Useful lead compounds are especially antibodies and peptides, and particularly intracellular antibodies expressed within the cell in a gene therapy context, which may be used as models for the development of peptide or low molecular weight therapeutics. In a preferred aspect of the invention, lead compounds and ABIN2/TPL-2/p105 or other target peptide may be co-crystallised in order to facilitate the design of suitable low molecular weight compounds which mimic the interaction observed with the lead compound.

Crystallisation involves the preparation of a crystallisation buffer, for example by mixing a solution of the peptide or peptide complex with a “reservoir buffer”, preferably in a 1:1 ratio, with a lower concentration of the precipitating agent necessary for crystal formation. For crystal formation, the concentration of the precipitating agent is increased, for example by addition of precipitating agent, for example by titration, or by allowing the concentration of precipitating agent to balance by diffusion between the crystallisation buffer and a reservoir buffer. Under suitable conditions such diffusion of precipitating agent occurs along the gradient of precipitating agent, for example from the reservoir buffer having a higher concentration of precipitating agent into the crystallisation buffer having a lower concentration of precipitating agent. Diffusion may be achieved for example by vapour diffusion techniques allowing diffusion in the common gas phase. Known techniques are, for example, vapour diffusion methods, such as the “hanging drop” or the “sitting drop” method. In the vapour diffusion method a drop of crystallisation buffer containing the protein is hanging above or sitting beside a much larger pool of reservoir buffer. Alternatively, the balancing of the precipitating agent can be achieved through a semipermeable membrane that separates the crystallisation buffer from the reservoir buffer and prevents dilution of the protein into the reservoir buffer.

In the crystallisation buffer the peptide or peptide/binding partner complex preferably has a concentration of up to 30 mg/ml, preferably from about 2 mg/ml to about 4 mg/ml.

Formation of crystals can be achieved under various conditions which are essentially determined by the following parameters: pH, presence of salts and additives, precipitating agent, protein concentration and temperature. The pH may range from about 4.0 to 9.0. The concentration and type of buffer is rather unimportant, and therefore variable, e.g. in dependence with the desired pH. Suitable buffer systems include phosphate, acetate, citrate, Tris, MES and HEPES buffers. Useful salts and additives include e.g. chlorides, sulphates and other salts known to those skilled in the art. The buffer contains a precipitating agent selected from the group consisting of a water miscible organic solvent, preferably polyethylene glycol having a molecular weight of between 100 and 20000, preferentially between 4000 and 10000, or a suitable salt, such as a sulphates, particularly ammonium sulphate, a chloride, a citrate or a tartarate.

A crystal of a peptide or peptide/binding partner complex according to the invention may be chemically modified, e.g. by heavy atom derivatization. Briefly, such derivatization is achievable by soaking a crystal in a solution containing heavy metal atom salts, or a organometallic compounds, e.g. lead chloride, gold thiomalate, thimerosal or uranyl acetate, which is capable of diffusing through the crystal and binding to the surface of the protein. The location(s) of the bound heavy metal atom(s) can be determined by X-ray diffraction analysis of the soaked crystal, which information may be used e.g. to construct a three-dimensional model of the peptide.

A three-dimensional model is obtainable, for example, from a heavy atom derivative of a crystal and/or from all or part of the structural data provided by the crystallisation. Preferably building of such model involves homology modelling and/or molecular replacement.

The preliminary homology model can be created by a combination of sequence alignment with any MAPKK kinase or NFκB the structure of which is known (including IκBα, Bauerle et al., (1998) Cell 95:729-731), secondary structure prediction and screening of structural libraries. For example, the sequences of ABIN2/TPL-2/p105 and a candidate peptide can be aligned using a suitable software program.

Computational software may also be used to predict the secondary structure of the peptide or peptide complex. The peptide sequence may be incorporated into the ABIN2/TPL-2/p105 structure. Structural incoherences, e.g. structural fragments around insertions/deletions can be modelled by screening a structural library for peptides of the desired length and with a suitable conformation. For prediction of the side chain conformation, a side chain rotamer library may be employed.

The final homology model is used to solve the crystal structure of the peptide by molecular replacement using suitable computer software. The homology model is positioned according to the results of molecular replacement, and subjected to further refinement comprising molecular dynamics calculations and modelling of the inhibitor used for crystallisation into the electron density.

### 5. Pharmaceutical Compositions

In a preferred embodiment, there is provided a pharmaceutical composition comprising a compound or compounds identifiable by an assay method as defined in the previous aspect of the invention.

A pharmaceutical composition according to the invention is a composition of matter comprising a compound or compounds capable of modulating the p105-stabilising activity of ABIN2 as an active ingredient. The active ingredients of a pharmaceutical composition comprising the active ingredient according to the invention are contemplated to exhibit excellent therapeutic activity, for example, in the treatment of tumours or other diseases associated with cell proliferation, infections and inflammatory conditions, when administered in amount which depends on the particular case. Dosage regima may be adjusted to provide the optimum therapeutic response. For example, several divided doses may be administered daily or the dose may be proportionally reduced as indicated by the exigencies of the therapeutic situation.

The active ingredient may be administered in a convenient manner such as by the oral, intravenous (where water soluble), intramuscular, subcutaneous, intranasal, intradermal or suppository routes or implanting (e.g. using slow release molecules). Depending on the route of administration, the active ingredient may be required to be coated in a material to protect said ingredients from the action of enzymes, acids and other natural conditions which may inactivate said ingredient.

In order to administer the active ingredient by other than parenteral administration, it will be coated by, or administered with, a material to prevent its inactivation. For example, the active ingredient may be administered in an adjuvant, co-administered with enzyme inhibitors or in liposomes. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin.

Liposomes include water-in-oil-in-water CGF emulsions as well as conventional liposomes.

The active ingredient may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as lecithin, by the maintenance of the required particle size in the case of dispersion and by the use of superfactants.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active ingredient in the required amount in the appropriate solvent with various of the other ingredients enumerated above, as required, followed by filtered sterilisation. Generally, dispersions are prepared by incorporating the sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredient is suitably protected as described above, it may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active ingredient may be incorporated with excipients and used in the form of ingestible tablets, buccal tablets, troches, capsules, elixirs, suspensions, syrups, wafers, and the like. The amount of active ingredient in such therapeutically useful compositions in such that a suitable dosage will be obtained.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active ingredient, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active ingredient may be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such as active material for the treatment of disease in living subjects having a diseased condition in which bodily health is impaired.

The principal active ingredients are compounded for convenient and effective administration in effective amounts with a suitable pharmaceutically acceptable carrier in dosage unit form. In the case of compositions containing supplementary active ingredients, the dosages are determined by reference to the usual dose and manner of administration of the said ingredients.

In a further aspect there is provided the active ingredient of the invention as hereinbefore defined for use in the treatment of disease. Consequently there is provided the use of an active ingredient of the invention for the manufacture of a medicament for the treatment of disease associated with NFκB induction or repression.

Moreover, there is provided a method for treating a condition associated with NFκB induction or repression, comprising administering to a subject a therapeutically effective amount of a compound or compounds identifiable using an assay method as described above.

The invention is further described, for the purpose of illustration only, in the following examples.

### Materials and Methods

cDNA Constructs.

For transient transfection experiments in 293 cells, all hemagglutinin (HA) epitope-tagged NF-κB1 p105 (Ha-p105) cDNAs were cloned into the pcDNA3 vector (Invitrogen). Deletion and point mutant versions of Ha-p105 and Ha-p50 have been described previously (1, 2, 24). For stable transfection of HeLa S3 cells, Ha-p105(S927A) was subcloned in the pMX-1 vector (Ingenius). Myc epitope-tagged p105 (Myc-p105) was generated by PCR and verified by DNA sequencing. Myc-tagged and untagged versions of TPL-2, kinase-inactive TPL-2(D270A) and TPL-2ΔC have been described previously (3). Myc-A20 was kindly provided by Nancy Raab-Traub (University of North Carolina, USA) (10).

Human ABIN-2 cDNA (Image clone 4287014) was obtained from the U.K. Human Genome Mapping Project resource centre (Cambridge). Wild type ABIN-2 was FLAG-tagged on its C-terminus (ABIN-2-FL) using PCR and cloned into the pcDNA3 vector (Invitrogen). PCR was also used to generate the following panel of ABIN-2 deletion mutants subcloned into pGEX-2T (Amersham Biosciences): GST-ABIN-2₁₋₄₂₉, GST-ABIN-2₁₋₈₉, GST-ABIN-2₁₋₁₀₈, GST-ABIN-2₁₋₁₂₉, GST-ABIN-2₁₋₁₉₃, GST-ABIN-2₁₋₂₅₀, GST-ABIN-2₉₀₋₂₅₀, GST-ABIN-2₁₃₀₋₂₅₀, GST-ABIN-2₁₉₄₋₂₅₀ and GST-ABIN-2₂₅₁₋₄₂₉. All constructs were verified by DNA sequencing.

### Recombinant Proteins, Peptides and Antibodies.

Glutathione S-transferase (GST) fusion ABIN-2 proteins were expressed at 30° C. in Escherichia coli BL21 (DE3) and purified by affinity chromatography on glutathione (GSH)-Sepharose 4B (Amersham Biosciences). Purity of GST fusion proteins was estimated to be >90% by Coomassie brilliant blue (Novex) staining after sodium dodecyl sulfate-polyacrylamide gel electrophoresis (SDS-PAGE). GST-p105₄₉₇₋₉₆₈ fusion protein has been described previously (2). GST-MEK1, GST-MEK1(K207A) and GST-ERK fusion proteins were kindly provided by Richard Marais (Cancer Research—U.K., London).

Antibodies to HA, Myc and FLAG (FL) epitope tags have been described previously (1). Anti-human p105C, anti-murine p105C, anti-human p105C and anti-murine p105N antibodies have also been described (7, 24). 70mer anti-TPL-2 antibody was raised in rabbits against a synthetic peptide corresponding to the C-terminal 70 amino acids of rat TPL-2 coupled to keyhole limpet hemocyanin (KLH; Pierce) and was used for immunoprecipitation of endogenous TPL-2 protein. A commercial anti-TPL-2 antibody (Santa Cruz; M20) was used to detect TPL-2 in Western blots. Anti-ABIN-2 antibody was raised in rabbits against GST-ABIN-2₂₅₁₋₄₂₉ fusion protein. Endogenous p100 was detected using a commercial anti-p100 antibody (UBI 05-361). Anti-MEK-1/2 and anti-phospho(S217/S221)-MEK-1/2 (phospho-MEK-1/2) antibodies were purchased from Cell Signaling Technology (USA). Tubulin was detected with the TAT-1 anti-α-tubulin MAb (kindly provided by Keith Gull, University of Manchester, U.K.) and actin using a commercial anti-actin MAb (Sigma). Tubulin and actin were used as loading controls for Western blots of cell lysates.

3×-HA peptide was synthesized by Pete Fletcher (Protein Structure, NIMR) and consisted of the sequence to which the 12CA5 anti-HA MAb was raised (YPYDVPDYA) triplicated, with three glycine residue spacers between each HA sequence. This was significantly more efficient for elution of affinity purified protein from 12CA5 MAb than a single copy peptide (data not shown).

### Cell Culture.

HeLa S3 and 293 cells were cultured in Dulbecco's modified Eagle's medium (DMEM; Invitrogen) supplemented with 10% fetal calf serum, 2 mM glutamine, penicillin (100 units/ml) and streptomycin (50 units/ml) (complete DMEM). Cells were maintained in a rapid growth phase prior to use in experiments.

For stable transfection of HeLa S3 cells, 7×10⁵ cells were plated in a 60-mm dish (Nunc) and, after 18 h in culture, transfected using a standard calcium phosphate method with 5 μg pMX-1 Ha-p105(S927A). Transfected cells were cultured for a further 48 h and then selected for neomycin resistance with 1 mg/ml G418 (Invitrogen). After 3-4 weeks, 48 clones were picked manually and then expanded. Five of these clones tested positive for expression of Ha-p105(S927A) protein by Western blotting. Clone C3.25, which expressed relatively high levels of Ha-p105(S927A) protein, was selected for preparative experiments. A clone transfected with empty pMX-1 vector (EV) was used as a control.

Bone marrow-derived macrophages (BMDMs) were prepared as described (30). Briefly, Balb/c bone marrow cells were plated at 2×10⁶ cells/ml in 10 ml of complete BMDM medium (RPMI 1640 (Invitrogen) plus 10% fetal bovine serum (FBS) and antibiotics supplemented with 10% L-cell conditioned medium) in 25-cm² tissue culture flasks (Nunc). After 24 h, non-adherent cells were then transferred to a 80 cm² tissue culture flask (Nunc) and another 10 ml of complete BMDM medium added. Flasks were then incubated for 3 d at 37° C., at which time a further 10 ml of complete BMDM medium was added. After a total of 7 d culture, adherent macrophages were harvested, replated and cultured in RMPI 1640 medium plus 0.5% FBS and antibiotics for a further 24 h before use in experiments. Over 95% of the resulting cell populations were macrophages as judged by flow cytometric analysis (data not shown).

Nf-κb1^{-/-} mice (26) were obtained from the Jackson Laboratories. BMDMs from these knockout mice and heterozygous littermates (6-10 weeks) were prepared as described above.

### Affinity Purification of Ha-p105(S927A).

20 ml cell pellets of C3.25 Ha-p105(S927A) and EV HeLa S3 cells were prepared by centrifugation from large scale suspension cell cultures (20 L). Cells were lysed in 10 volumes of ice cold buffer A (1% NP-40, 50 mM Tris—pH7.5; 150 mM NaCl, 1 mM EDTA, 1 mM EGTA, 20 mM NaF, 1 mM Na₃VO₄, 1 mM Na₄P₂O₇ plus a mixture of protease inhibitors (Roche Molecular Biochemicals)). All subsequent steps were carried out at 4° C. Lysates were centrifuged at 20,000 g for 30 min and passed through a glass fiber filter (Nalgene, 189-2000) to remove lipids. Filtered lysates were re-centrifuged at 100,000 g for a further 30 min and RQI RNAase-free DNAase (Promega, M610A; 1/1000 stock) added.

Lysates were pre-cleared of non-specific binding proteins firstly by two sequential batch incubations (overnight and 2 h) with 1 ml aliquots of protein-A Sepharose beads (Amersham Biosciences). A further 3 h pre-clear was then performed by batch incubation with 0.5 ml of protein-A Sepharose coupled to 3.5 mg of purified mouse IgG (Sigma technical grade). Lysates were finally pre-cleared by passing under gravity through 5 ml of protein-A Sepharose packed in a disposable column (Biorad 732-1010).

Ha-p105(927A) protein was affinity purified by incubating pre-cleared lysate overnight with 0.5 ml of protein-A Sepharose beads coupled to 3.5 mg of 12CA5 anti-HA MAb. The suspension of the 12CA5 beads in lysate was then transferred to a disposable column (Biorad 732-1010) and washed with 100 column volumes of buffer A (flow rate<1 ml/min). Washed beads were transferred to siliconized tubes (0.5 ml; Bioquote), which were used in all subsequent steps, and excess buffer removed. To elute bound protein, 250 μl of peptide elution buffer (3×-HA peptide dissolved in 50 mM Tris—pH7.5, 150 mM NaCl, 0.05% NP-40) was added and beads incubated for 15 min with rotation. Eluate was removed from centrifuged beads and transferred to new tubes. This process was repeated five times, eluates combined (total volume=1.5 ml), snap frozen and stored at −70° C.

One third of the 3×-HA peptide eluate (0.5 ml) was diluted ½ in buffer A and pre-cleared twice (overnight and for 1 h) by incubation with 25 μl of rabbit IgG (Sigma) coupled to protein-A Sepharose beads (0.5 mg/ml). Eluted Ha-p105(S927A) protein was then re-immunoprecipitated by incubation for 5 h with 40 μl of anti-p105C antibody IgG coupled to protein-A Sepharose (0.35 mg/ml). Beads were washed five times with buffer A, once with water and bound protein eluted with 50 μl of 0.1M glycine—pH3.0, 0.05% NP-40. Low pH elution was repeated five times, eluates combined and neutralized with 1/10 volume of 1M tris—pH8.0. 1/200 of the eluate (370 μl), containing purified Ha-p105(S927A) and associated proteins, was resolved by 10%-acrylamide SDS-PAGE and isolated proteins revealed by silver staining (14). The remainder of the eluate was snap frozen and stored at −70° C. until processing for mass spectroscopic analysis.

### Mass Spectroscopic Analysis.

270 μl of affinity purified eluate containing Ha-p105(S927A) was concentrated to 30 μl using a Microcon YM-10 (Millipore) and then mixed with 7.5 μl of 4× sample buffer. Isolated proteins were resolved by 10%-acrylamide SDS-PAGE and revealed by staining with colloidal Coomassie brilliant blue (Novex). The stained gel was not scanned prior to excision of protein bands to minimize handling and potential introduction of contaminating keratins. Excised protein bands were reduced with 20 mM dithiothreitol and alkylated with 5 mM iodoacetamide. Bands were then dried and reswollen in 2 ng/μl trypsin (modified sequencing grade, Promega) in 5 mM ammonium bicarbonate. After overnight digestion at 32° C., the supernatant was acidified by addition of 1/10^{th} volume of trifluoroacetic acid.

Peptide mass fingerprinting was performed using a Reflex III MALDI time-of-flight mass spectrometer (Bruker Daltonik GmbH, Bremen, Germany) equipped with a nitrogen laser and a Scout-384 probe to obtain positive ion mass spectra. 0.4 μl of digestion supernatant was analyzed after desalting with water on the matrix surface. Peptide mass fingerprints were searched against the non-redundant protein database at the National Center for Biotechnology Information (NCBI) using the MASCOT program.

### Protein Analyses.

To analyze interactions with endogenous ABIN-2 protein, HeLa S3 cells were plated at 5×10⁶ cells per 60-mm diameter dish (Nunc) and cultured overnight. Cells were then washed in phosphate-buffer saline (PBS) and lysed in 1 ml of buffer A. Lysates were cleared of particulate matter by centrifugation at 100,000 g for 10 min. Immunoprecipitation and Western blotting of proteins was carried out as described previously (17). However, two additional steps were taken to minimize detection of 1 g heavy chain in immunoprecipitates, which co-migrates with both ABIN-2 and TPL-2. Firstly, all antibodies were covalently coupled to protein-A Sepharose using dimethylpimelimidate (25) and immunoprecipitated protein was eluted using a low pH buffer (50 μl; 0.2M glycine—pH2.5, 0.05% NP-40). Eluate was neutralized by addition of 10 μ1 M Tris—pH8 and then mixed with an equal volume of 2×SDS-PAGE sample buffer. Secondly, Western blot membranes were blocked with protein A (5 μg/ml; Sigma) prior to probing with primary rabbit antibody. Bound antibody was then revealed with protein-A coupled to horse radish peroxidase (Amersham Biosciences) and enhanced chemiluminescence (Amersham Biosciences).

293 cells (3×10⁵ cells per 60-mm diameter Nunc dish) were transiently transfected using Lipofectamine (Invitrogen) and cultured for a total of 48 h, as described previously (2). For protein association experiments, cell lysates were prepared using 1% NP-40 buffer A. Immunoprecipitation and Western blotting was carried out as described previously (17). In pulse-chase experiments, cells were washed in PBS after 24 h culture and then incubated for 45 min in methionine-cysteine-free minimal Eagle medium (Sigma) plus 0.5% FBS. Cells were pulse-labeled with 2.65 MBq of [³⁵S]methionine-[³⁵S]cysteine (Pro-Mix; Amersham Biosciences) for 30 min and chased for the indicated times in DMEM plus 2% fetal calf serum. Lysis was carried out using buffer A supplemented with 0.5% deoxycholate and 0.1% SDS (radio-immunoprecipitation assay buffer; RIPA). TPL-2 was isolated using 70mer anti-TPL-2 antibody and labeled bands revealed by auto-radiography after 10%-acrylamide SDS-PAGE.

BMDMs were plated in 60-mm dishes (3×10⁶ cells; Nunc) and cultured for 18 h culture cells prior to lysis in 1% NP-40 buffer A. 35-mm dishes (1×10⁶ cells; Nunc) were used in experiments in which cells were stimulated with LPS (1 g/ml; S. minessota; Alexis Biochemicals). Where indicated, cells were preincubated for 30 min with MG132 proteasome inhibitor (40 μM; Biomol) or DMSO vehicle control prior to LPS stimulation. Proteins were removed from lysates by immunoprecipitation with pre-clearing antibody or pre-immune rabbit IgG as a control, both covalently coupled to protein-A Sepharose. In some experiments, pre-cleared lysates were re-immunoprecipitated overnight with the indicated specific antibody. Lysates and re-immunoprecipitated proteins were resolved by 10%-acrylamide SDS-PAGE and Western blotted.

For pulldown assays with GST-ABIN-2 fusion proteins, 2 μg of recombinant protein was added to ultracentrifuged lysate of transfected 293 cells. For some of these experiments, 1% Brij-58 was used as the detergent component of buffer A used for lysis, rather than 1% NP-40, as indicated in the Figure legends. Lysates were incubated overnight with mixing and fusion proteins affinity isolated by addition of 10 μl of glutathione Sepharose 4B beads (Amersham Biosciences) and incubation for a further 30 min. Beads were then washed extensively in buffer A (1% NP-40 or 1% Brij-58, as appropriate) and isolated protein analyzed by Western blotting. To investigate whether ABIN-2 could bind to the C-terminal half of p105, ABIN-2-FL was synthesized from its expression vector and labeled with [³⁵S]methionine (Amersham Bioscience) by cell-free translation (25 μl reaction volume; Promega TNF-coupled rabbit reticulocyte system). Translated protein was diluted in 1 ml of 1% NP-40 buffer A and then incubated with 5 μg of GST-p105₄₉₇₋₉₆₈ fusion protein bound to glutathione Sepharose 4B (Amersham Bioscience). After an overnight incubation at 4° C., beads were extensively washed with buffer A and bound [³⁵S]-labeled protein visualized by autoradiography after 10%-acrylamide SDS-PAGE.

To demonstrate binding of ABIN-2 to the isolated TPL-2 C-terminus, 30% g of biotinylated TPL-2398467 peptide (2) was incubated for 2 h with lysates (1% NP-40 buffer A) of 293 cells transfected with a plasmid encoding ABIN-2-FL. TPL-2 peptide was captured on streptavidin-agarose beads (Sigma) which were then washed extensively with buffer A. Isolated protein was resolved by 10%-acrylamide SDS-PAGE and Western blotted.

### RNA Interference.

RNA interference was used to deplete HeLa S3 and 293 cells of endogenous ABIN-2. Small interfering RNAs (siRNAs) were synthesized by Xeragon Inc. (USA). The sequences of the ABIN-2 siRNAs used were: (sense) GUAUUUGGCCGCCGACGCAd(TT) and (antisense) UGCGUCGGCGGCCAAAUACd(TT). Commercial control siRNAs (Xeragon; 1022076) were used to confirm the specificity of the ABIN-2 siRNAs effects.

For gene knockdown experiments, HeLa S3 cells (5×10⁵) or 293 cells (2×10⁵) were plated in 60-mm diameter dish (Nunc) and cultured for 12-16 h in complete DMEM medium without antibiotics. Cells were transfected with siRNAs (0.4 nmol per well) using Lipofectamine 2000 (Invitrogen) according to the manufacturer's instructions. After 24 h culture, cells were re-transfected with siRNAs and then re-cultured for a further 48 h. Protein expression was analyzed by Western blotting of cell lysates. Semi-quantitative reverse transcription-PCR(RT-PCR) of TPL-2 mRNA was performed by utilizing the Qiagen OneStep RT-PCR (RT-PCR) kit. Total RNA was isolated from cells using the Qiagen RNA easy kit. The TPL-2 primer pairs used were as follows: (5′)-primer, 5′-ACGCTAGTCGACTCACCTGTACGTCAGCTTCCACGG-3′; (3′)-primer, 5′-GCC CAG GGG ATC CGA ATG GAG TAC ATG AGC ACC G-3′. The 18rRNA loading control oligonucleotides used were: (5′) 5′-GGCGGCTTGGTGACTCTAGATA-3′ and (3′) 5′-GCTCGGGCCTGCTTT GAACAC-3′.

Semi-quantitative RT-PCR was also used to quantify ABIN-2 mRNA in wild type and NF-κB1-deficient 3T3 fibroblasts using the above methodology and the following primer pairs: (5′)-primer, 5′-CCATGTCGTCTGGGGACGCAA-3′ and (3′)-primer, 5′-TGGCAGCACTCAGACAGGTGC-3′.

### Mek Kinase Assays

To assay MEK kinase activity of endogenous TPL-2, BMDMs (8×10⁶) were plated in 90-mm dishes (Nunc). After 18 h in culture, cells were stimulated with LPS for 15 min and then lysed in kinase lysis-buffer (Buffer A containing 0.5% NP-40, 5 mM sodium O-glycerophosphate and 0.1% 2-mercaptoethanol). Lysates were immunoprecipitated for 4 h with anti-TPL-2 antibody coupled to Protein-A Sepharose and beads washed four times in kinase lysis-buffer and twice in kinase buffer (50 mM Tris [ph 7.5], 150 mM NaCl, 5 mM β-glycerophosphate, 2 mM dithiothreitol, 0.1 mM Na₃VO₄, 10 mM MgCl₂, 1 mM EGTA, 0.03% Brij-35). The beads were then resuspended in 25 μl kinase buffer supplemented with 1 mM ATP, 6.5 μg/ml GST-MEK and 100 μg/ml GST-ERK and incubated for 30 min at room temperature. After centrifugation, 2 μl of the supernatant was added to 48 μl of kinase buffer containing 0.33 mg/ml myelin basic protein (MBP, Sigma), 0.1 mM ATP and 2.5 μCi [γ-³²P]ATP (Amersham Biosciences) and incubated at room temperature for 10 min. The assay was terminated by adding 50 μl×SDS-PAGE sample buffer and labeled MBP revealed by autoradiography after 12.5%-acrylamide SDS-PAGE. Immunoprecipitated protein was eluted from anti-TPL-2 antibody beads with 0.2M glycine pH2.5, resolved 10%-acrylamide SDS-PAGE and Western blotted.

To test the effect of ABIN-2-FL co-expression on TPL-2 MEK kinase activity, Myc-TPL-2 was isolated by immunoprecipitation from lysates of co-transfected 293 cells, as described above. After washing, beads were resuspended in 50 μl of kinase buffer containing 1 mM ATP plus 1 μg of kinase inactive GST-MEK1(K₂₀₇A) and incubated at 30 min at room temperature. The supernatant was removed, mixed with an equal volume of 2×SDS-PAGE sample buffer and Western blotted after 10%-acrylamide SDS-PAGE. MEK phosphorylation was determined by probing blots with anti-phospho-MEK-1/2 antibody. Immunoprecipitated Myc-TPL-2 was eluted with SDS-PAGE sample buffer from the remaining anti-Myc MAb beads and quantified by Western blotting.

### EXAMPLE 1

### Affinity Purification of Ha-p105(S927A)

To more fully understand the function and regulation of NF-κB1 p105, affinity purification was used to identify novel p105-associated proteins. To do this, HeLa S3 cells were stably transfected with a vector encoding Ha-p105(S927A) and the C3.25 clone selected which expressed relatively high levels of the transfected protein. Ha-p105(S927A) contains a mutation of a critical serine residue in the p105 PEST region phosphorylated by the IKK complex and is thus resistant to signal-induced proteolysis (24). Higher levels of Ha-p105(S927A) were obtained than wild type Ha-p105 (data not shown), presumably due to proteolysis of the latter protein triggered by constitutive IKK activity in HeLa S3 cells.

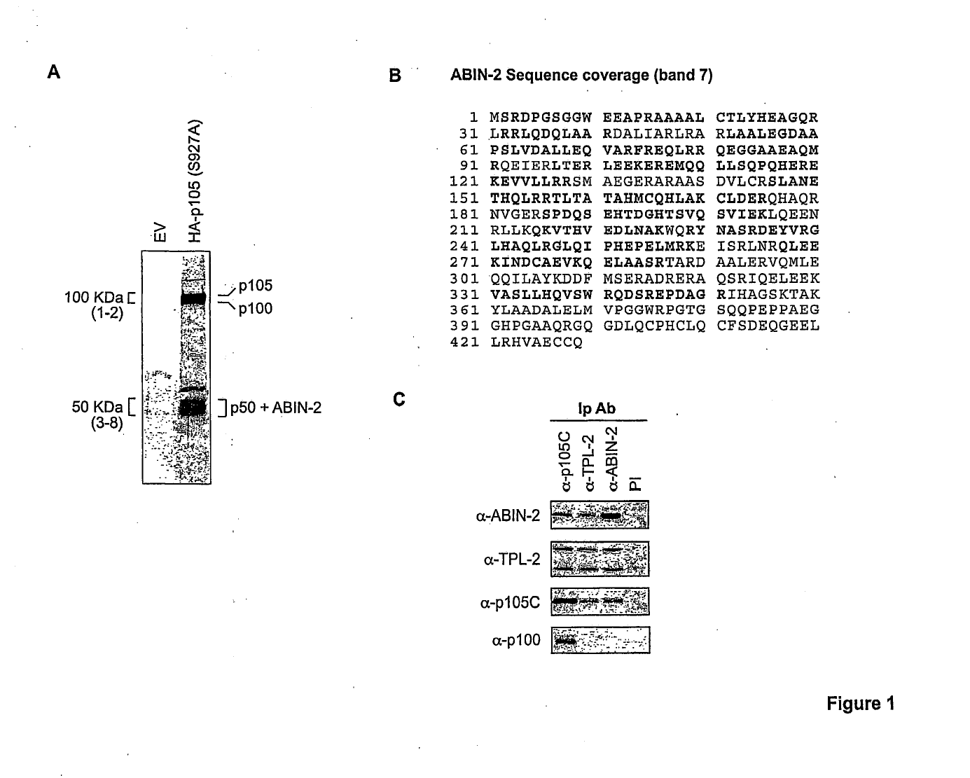

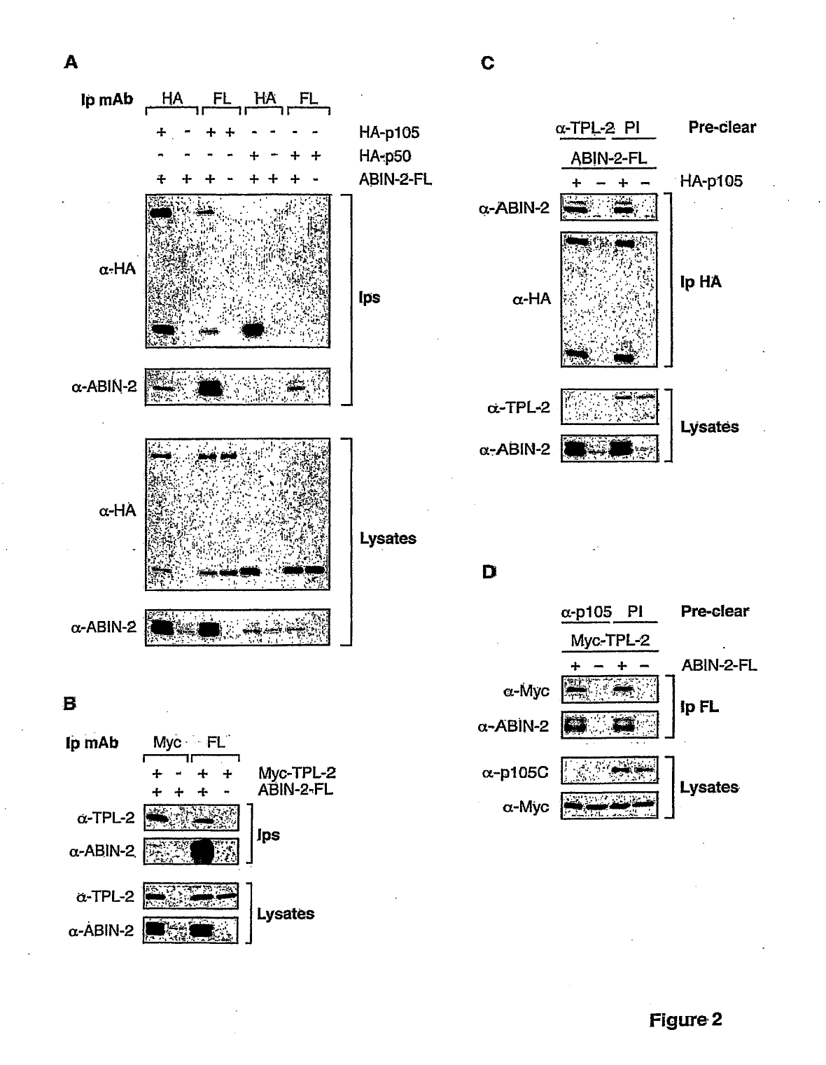

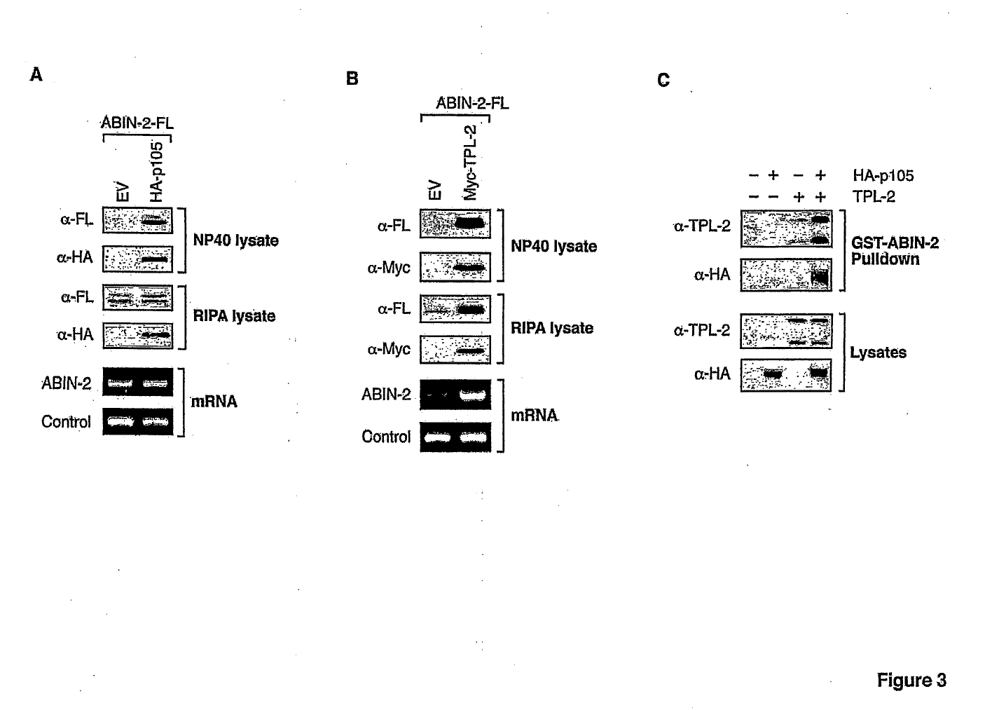

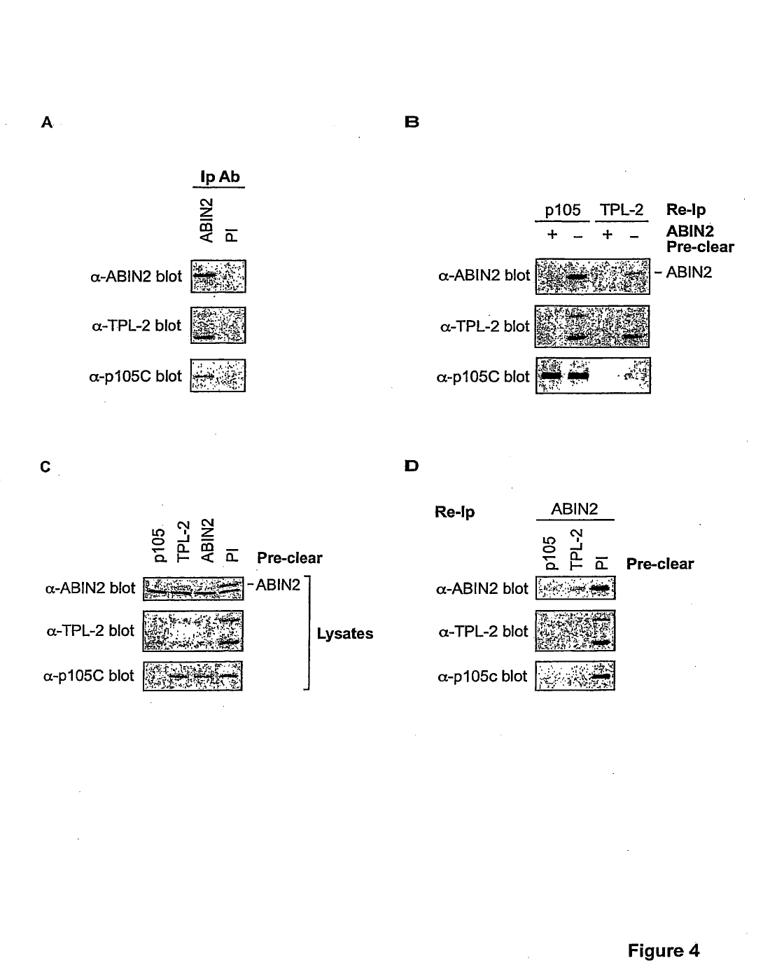

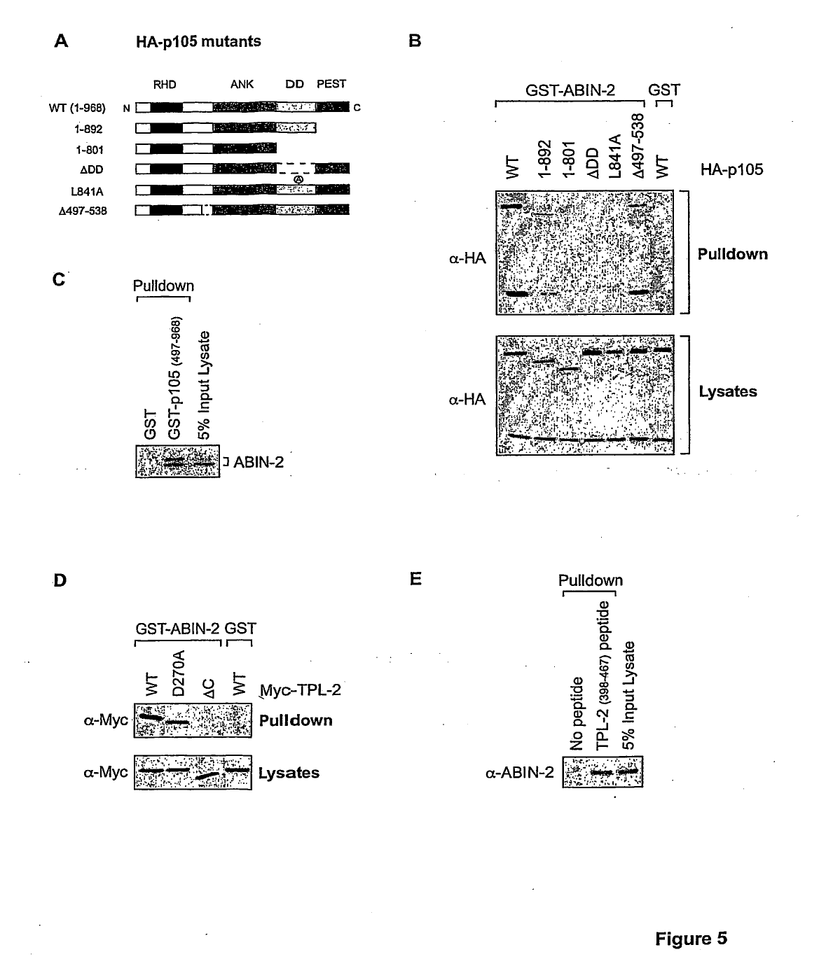

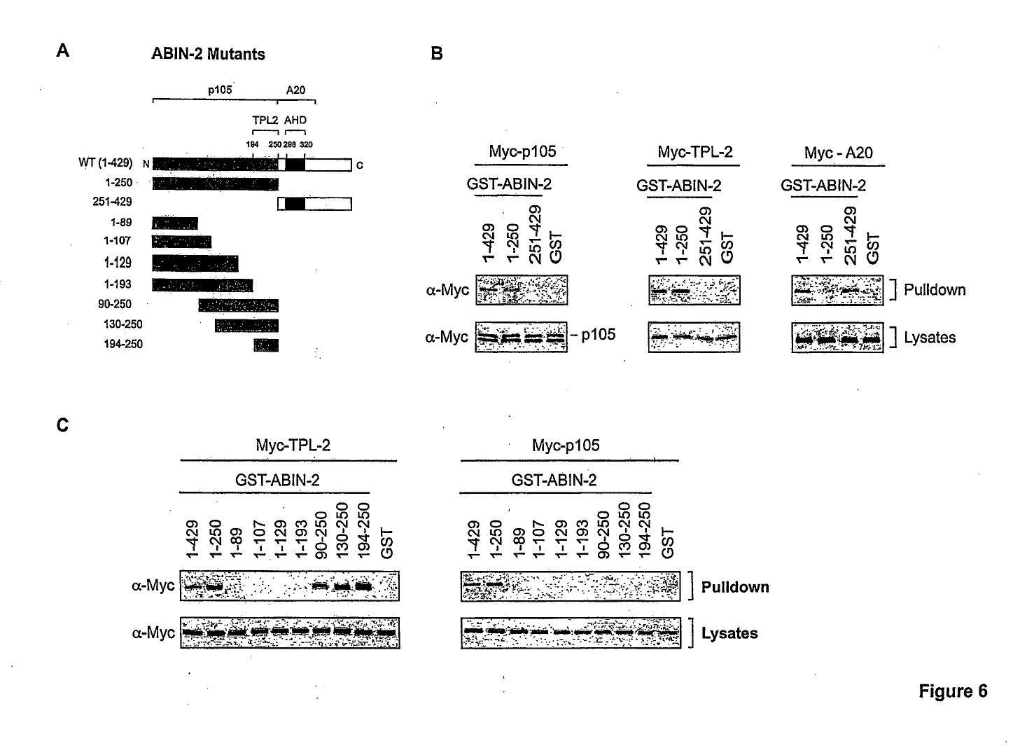

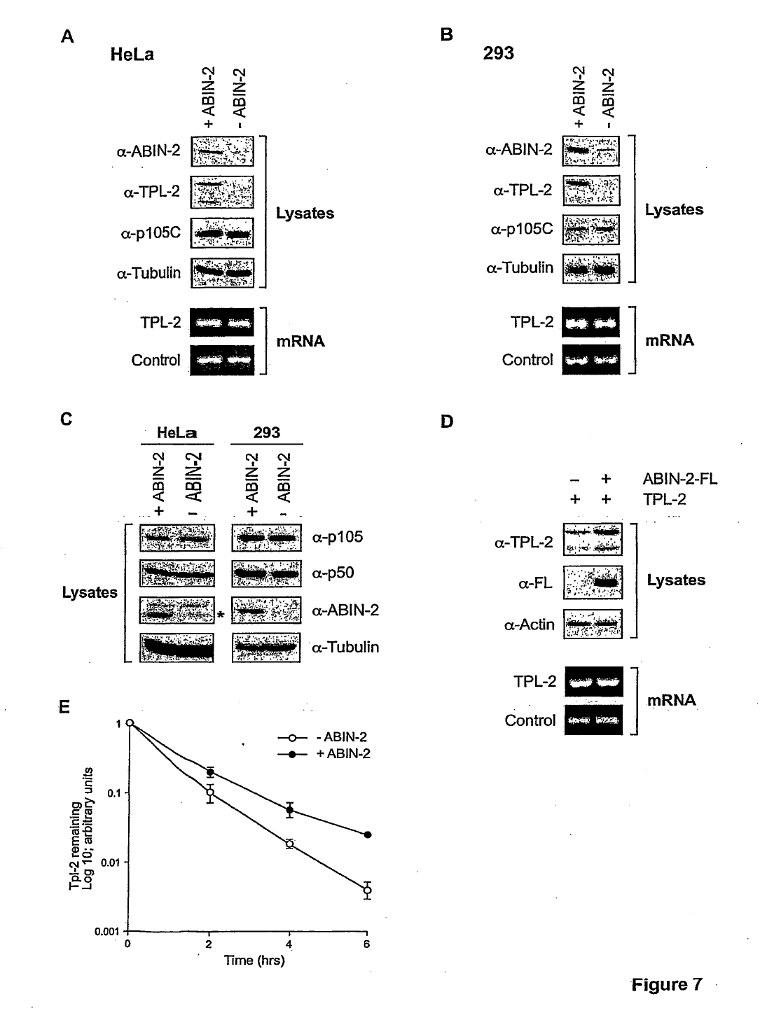

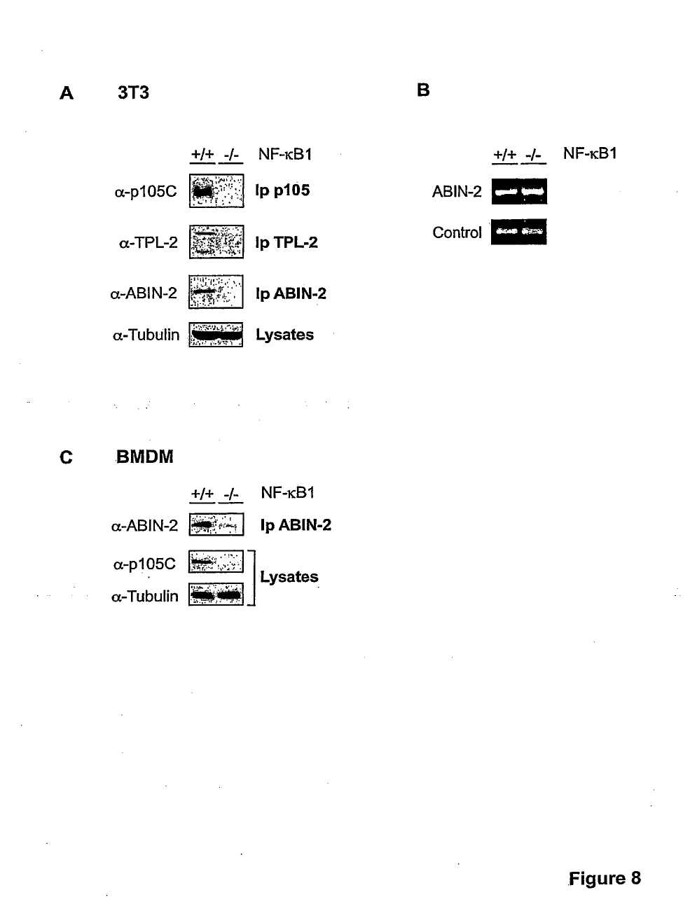

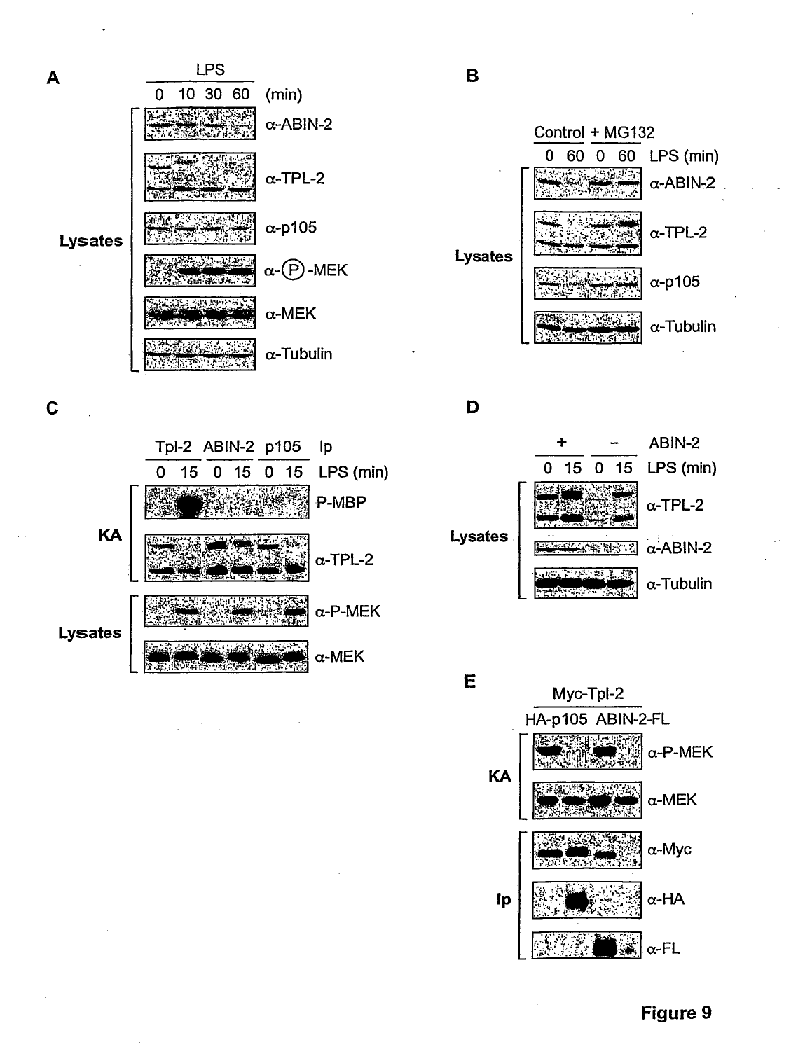

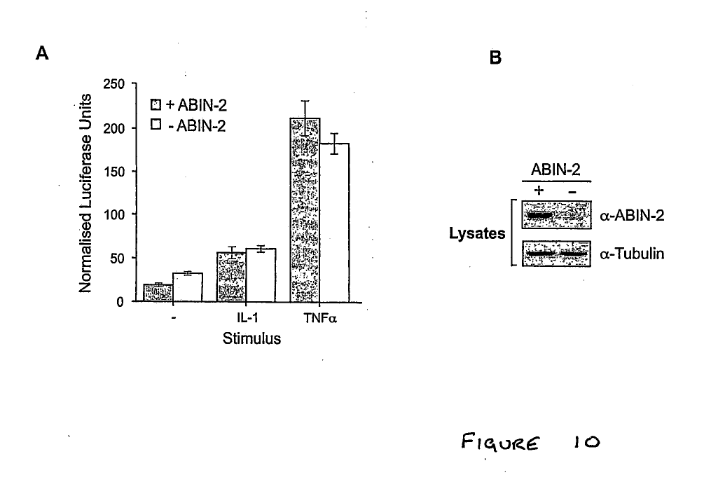

A two step, sequential affinity purification methodology was used to isolate Ha-p105(S927A) and associated proteins from a large scale suspension culture of C3.25 cells. Protein was first isolated from cell lysates using anti-HA MAb covalently linked to protein-A Sepharose beads. Bound protein was specifically eluted by incubation of washed beads with a 3×-HA peptide and then re-immunoprecipitated using an antibody directed against the C-terminus of p105. Re-immunoprecipitated protein was eluted from beads using a low pH buffer and 1/200 resolved by SDS-PAGE. Silver staining revealed a number of bands which were isolated from lysates of C3.25 cells but not from an equivalent number of control cells stably transfected with empty vector (EV) (FIG. 1A). Western blotting demonstrated that the major bands at approximately 100 kDa and 50 kDa co-migrated with Ha-p105 and Ha-p50, respectively (data not shown).

To identify isolated proteins, a fraction of the remaining eluted protein was concentrated and then resolved by 10%-acrylamide SDS-PAGE. Only the major bands of approximately 100 Da and 50 kDa were sufficiently abundant to be visualized by colloidal Coomassie blue staining, permitting further analysis (data not shown). The 100 Da region was excised in two adjacent slices (band 1 and band 2), whereas the 50 kDa region was excised in five adjacent slices (bands 3-8). Isolated protein bands were subjected to in-gel digestion and aliquots of the digest supernatants analyzed by MALDI mass spectroscopy. Masses of the resulting protonated peptides were used to search the NCBI non-redundant database. Band 1 was identified as NF-κB1 p105, whereas band 2 was found to contain both NF-κB1 p105 and NF-κB2 p100. NF-κB1 p50 was identified as the major component of bands 3 and 4. Bands 5-8 were found to contain both NF-κB1 p50 and also A20-binding inhibitor of NF-κB activation-2 (ABIN-2; (29)) which has not previously been linked to p105. These data indicate that NF-κB1 p50/Ha-p50, NF-κB2 p100 and ABIN-2 co-purify with Ha-p105(S927A) and suggest that ABIN-2 is a novel p105-associated protein.

### EXAMPLE 2

### ABIN-2 Specifically Associates with both p105 and TPL-2

Having identified ABIN-2 as a protein that co-purifies with stably overexpressed Ha-p105(S927A), it was important to determine whether ABIN-2 also interacted with p105 at physiological levels of both proteins. The endogenous proteins were immunoprecipitated from lysates of HeLa S3 cells and the isolated proteins Western blotted. ABIN-2 specifically co-purified in anti-p105C immunoprecipitates and conversely p105 co-purified in anti-ABIN-2 immunoprecipitates (FIG. 1C). Importantly, NF-κB2 p100, which is closely related to p105, did not co-immunoprecipitate with ABIN-2 (FIG. 1C) confirming the specificity of the p105/ABIN-2 association. As previously reported (21, 22), however, p100 was found to co-immunoprecipitate with p105, consistent with its co-purification with Ha-p105(S927A) (FIG. 1A). Thus, endogenous ABIN-2 specifically copurifies with p105 in a complex distinct from that containing p100.

Our previous studies demonstrated that the MAP 3-kinase TPL-2 is stoichiometrically associated with p105 in HeLa cells (3). Interestingly, immunoprecipitation of HeLa S3 lysates with anti-BOS TPL-2 antibody revealed that ABIN-2 specifically co-purified with TPL-2 and, conversely, TPL-2 was specifically co-purified in anti-ABIN-2 immunoprecipitates (FIG. 1C).

Since p105 and TPL-2 are associated with one another, the previous experiments did not distinguish whether ABIN-2 interacts directly with both p105 and TPL-2 or with only one of these proteins. To address this question, 293 cells were transiently co-transfected with plasmids encoding C-terminally FLAG-tagged ABIN-2 (ABIN-2-FL) and either HA-tagged p105 (Ha-p105) or Myc-tagged TPL-2 (Myc-TPL-2). Immunoprecipitation from transfected cell lysates and Western blotting revealed that ABIN-2-FL specifically co-immunoprecipitated with both Ha-p105 and Myc-TPL-2 (FIGS. 2 A and B). In contrast, similar experiments indicated that ABIN-2-FL did not associate with either Ha-p50 (FIG. 2A), the processed product of p105, or Ha-p100 (data not shown).

It was possible that ABIN-2-FL complexing with Ha-p105 was mediated via the association of endogenous TPL-2 with Ha-p105. Therefore, TPL-2 was removed from lysates of 293 cells cotransfected with vectors encoding ABIN-2-FL and Ha-p105 by sequential immunoprecipitation with anti-TPL-2 antibody. Removal of endogenous TPL-2 from 293 cell lysates did not reduce the level of ABIN-2-FL which co-immunoprecipitated with Ha-p105 (FIG. 2C). Similarly, immunodepletion of endogenous p105 with anti-p105C antibody did not alter binding of ABIN-2-FL to Myc-TPL-2 (FIG. 2D). Thus, ABIN-2-FL can interact independently with Ha-p105 and Myc-TPL-2.

Ha-p105 Increases the Solubility of Co-Expressed ABIN-2-FL in NP-40 Lysis Buffer

In the course of the previous experiments, it was noticed that the steady-state levels of transfected ABIN-2-FL detected by Western blot analysis of 1% NP-40 extracts were dramatically increased by co-expression with Ha-p105 (FIGS. 2A and 3A—upper panels). By comparison with lysis using a more stringent buffer containing ionic detergents (RIPA) and quantitation of ABIN-2-FL transcription by semi-quantitative PCR (FIGS. 2 A and 3A—lower panels), it was apparent this difference was due to increased extraction of ABIN-2-FL with 1% NP-40 buffer when co-expressed with Ha-p105, rather than increased production of ABIN-2-FL protein (FIG. 3A). Sub-cellular fractionation experiments indicated that this was not due to alteration in the localization of ABIN-2-FL (data not shown). Rather, the data suggest that Ha-p105 binding increases ABIN-2-FL solubility.

Steady-state levels of ABIN-2-FL were also dramatically increased by co-expression with Myc-TPL-2 (FIGS. 2B and 3B—upper panels). However, this increase was evident after cell extraction with 1% NP-40 buffer A or RIPA, and semi-quantitative PCR indicated that this was due to Myc-TPL-2 inducing production higher levels of ABIN-2-FL mRNA (FIG. 3B, lower panels). Kinase inactive Myc-TPL-2(D270A), which could bind to ABIN-2 (FIG. 5D), had little effect on steady-state levels of co-expressed ABIN-2-FL protein detected in cell lysates (data not shown).

ABIN-2 Forms a Ternary Complex with p105 and TPL-2.

In HeLa cells, the majority of TPL-2 is complexed with p105 (3). To determine whether ABIN-2 can interact with a p105/TPL-2 complex rather than p105 or TPL-2 alone, GST-ABIN-2 fusion protein was used as an affinity ligand to isolate Ha-p105 and TPL-2 from lysates of transiently transfected 293 cells. When expressed individually, GST-ABIN-2 interacted with TPL-2 but not Ha-p105 (FIG. 3C). However, co-expression of TPL-2 and Ha-p105, to allow formation of a TPL-2/Ha-p105 complex in vivo, facilitated GST-ABIN-2 pulldown of Ha-p105 and the level of Myc-TPL-2 isolated was significantly increased. These data indicate that GST-ABIN-2 preferentially forms a ternary complex with Ha-p105 and TPL-2.

Although GST-ABIN-2 did not form stable complexes with Ha-p105 (FIG. 3C) or Myc-p105 (data not shown) when extracted from cells using 1% NP40 detergent, interaction was clearly detected with Ha-p105 when cell lysates were prepared using the milder detergent Brij-58 (see FIG. 5B). In contrast, TPL-2 or Myc-TPL-2 bound to GST-ABIN-2 after extraction with either detergent (FIGS. 3C and 6B). These data suggest that the affinity of GST-ABIN-2 for TPL-2 is greater than that for Ha-p105.

The Majority of Endogenous ABIN-2 is Associated with TPL-2/p105 Complexes.

In macrophages, TPL-2 is essential for LPS activation of the MEK/ERK MAP kinase pathway (9) and must interact with p105 to maintain its steady-state expression (31). To determine whether ABIN-2 is associated with TPL-2 and p105 in this physiologically relevant cell type, lysates were prepared from bone marrow-derived macrophages (BMDMs). Immunoprecipitation and Western blot analysis confirmed that both p105 and TPL-2 co-purified with ABIN-2 in these cells (FIG. 4A).

Next, it was investigated whether an ABIN-2/TPL-2/p105 ternary complex exists in BMDMs. To do this, endogenous ABIN-2 was depleted from BMDM lysates by serial immunoprecipitation with anti-ABIN-2 antibody. ABIN-2 immunodepletion removed the majority of TPL-2 detected directly in cell lysates or after re-immunoprecipitation with anti-TPL-2 or anti-p105 antibodies (FIG. 4 B and C). Immunodepletion of p105 using anti-p105C antibody also removed a substantial fraction of detectable ABIN-2 (FIG. 4C) and cleared TPL-2 from lysates, similar to earlier experiments with HeLa cells (3). Together these data imply that TPL-2 is present in a complex with both p105 and ABIN-2 in BMDMs. Consistent with this conclusion, anti-TPL-2 antibody immunodepletion removed only a small fraction of total p105 from cell lysates (FIG. 4C) but the majority of ABIN-2-associated p105 (FIG. 4D). Significantly, immunodepletion of TPL-2 removed a substantial fraction of total ABIN-2 detected directly in lysates (FIG. 4 C) or after re-immunoprecipitation with anti-ABIN-2 antibody (FIG. 4D). Thus the majority of ABIN-2 is associated with p105/TPL-2 complexes and the majority of TPL-2 is associated with ABIN-2 in macrophages. However, only a small fraction of total cellular p105 participates in these ternary complexes (FIG. 4C).

### EXAMPLE 3

### Mapping the Regions Involved in Interaction of ABIN-2 with p105 and TPL-2

To further characterize the association between ABIN-2 and the p105/TPL-2 complex, the interacting regions of each protein were mapped. To analyze the interaction of p105 with ABIN-2, 293 cells were transiently transfected with vectors encoding a panel of deletion and point mutants of Ha-p105 (see FIG. 5A; (2)). Lysates, prepared using buffer A containing 1% Brij-58 detergent, were then incubated with GST-ABIN-2₁₋₄₂₉ fusion protein in a pulldown assay. Western blotting of isolated proteins demonstrated binding of GST-ABIN-2₁₋₄₂₉ to wild type Ha-p105 (FIG. 5B). Binding was significantly decreased by deletion of the PEST region (Ha-p105₁₋₈₉₂) and completely lost by further deletion to remove the death domain (DD; Ha-p105₁₋₈₀₁). Binding was also abrogated by internal deletion of the DD (Ha-p105ADD) or functional inactivation of the DD by point mutation (Ha-p105L841A; (2)). Deletion of the region N-terminal to the ankyrin repeats that binds to the TPL-2 C-terminus (Ha-p105_{Δ497-538}) also slightly reduced binding to GST-ABIN-2. Thus, the p105 DD is essential for ABIN-2 binding to p105 but optimal association also requires the PEST region and, to a lesser extent, p105 residues 497-538. Previous experiments have indicated that maximal interaction of TPL-2 with p105 requires the p105 DD and p105 residues 497-538 but does not involve the p105 PEST region (2). Thus, ABIN-2 interacts with similar regions of p105 to TPL-2 but not identical. A pulldown experiment with GST-p105₄₉₇₋₉₆₈ protein (2) confirmed that the isolated C-terminal half of p105 was sufficient for binding to ABIN-2-FL (FIG. 5C).

Activation of the oncogenic potential of TPL-2 requires deletion of its C-terminus (5). In an earlier study by this laboratory, it was demonstrated that the TPL-2 C-terminus forms a high affinity interaction with a region N-terminal to the ankyrin repeats of p105 (2). To determine whether the TPL-2 C-terminus is also involved in interaction with ABIN-2, 293 cells were transfected with vectors encoding Myc-TPL-2 or Myc-TPL-2ΔC. Pulldowns assays with revealed that the TPL-2 C-terminus was required for interaction with GST-ABIN-2₁₋₄₂₉ (FIG. 5D). Myc-TPL-2(D270A) bound GST-ABIN-2₁₋₄₂₉ to a similar degree to wild type Myc-TPL-2 indicating that its kinase activity is not required for TPL-2/ABIN-2 interaction. In contrast, previous experiments have indicated that the D270A mutation significantly decreases the interaction of TPL-2 with p105 (2). A pulldown assay with biotinylated TPL-2₃₉₈₋₄₆₇ peptide (2) coupled to streptavidin-agarose beads demonstrated that the isolated TPL-2 C-terminus is sufficient for binding to ABIN-2-FL (FIG. 5E).

A fragment comprising amino acids 251-429 of ABIN-2 contains both it's A20 binding and NF-κB inhibitory functions (29). To determine whether p105 and TPL-2 interact with the same region of ABIN-2, GST-ABIN-2₁₋₂₅₀ and GST-ABIN-2₂₅₁₋₄₂₉ fusion proteins were assayed for their ability to interact with Myc-p105 or Myc-TPL-2 in pulldown assays. Both Myc-p105 and Myc-TPL-2 bound to GST-ABIN-2₁₋₂₅₀ but not GST-ABIN-2₂₅₁₋₄₂₉ (FIG. 6B). In contrast, Myc-A20 interacted with GST-ABIN-2₂₅₁₋₄₂₉ but not GST-ABIN-2₁₋₂₅₀ (FIG. 6B), consistent with previously published results (29). Thus, p105 and TPL-2 interact with a different part of ABIN-2 to A20.

To more finely map the region of the ABIN-2 N-terminal half involved in interaction with Myc-TPL-2 and Myc-p105, additional GST-ABIN-2 fusion proteins were generated (see FIG. 6A). Pulldown experiments revealed that Myc-TPL-2 bound to a region containing amino acids 194-250 of ABIN-2 (FIG. 6C, left panel). Homology searches did not reveal any close similarity between this region and other proteins in the database. In contrast, Myc-p105 only detectably interacted with the entire 1-250 fragment of ABIN-2 (FIG. 6C, right panel). These data indicate that the interactions of p105 and TPL-2 with ABIN-2 are distinct, consistent with the earlier conclusion that p105 and TPL-2 can independently bind to ABIN-2 (FIGS. 3 A and B).

### EXAMPLE 4

### ABIN-2 is Required to Maintain the Metabolic Stability of TPL-2 Protein

Previous studies have indicated that p105 binding to TPL-2 is required to stabilize TPL-2 protein and maintain its steady-state levels in both macrophages and fibroblasts (2, 31). Since TPL-2 is present in a ternary complex with p105 and ABIN-2 in cells, it was of interest to determine whether TPL-2 protein stability was also influenced by ABIN-2 binding. To investigate this, siRNA-mediated gene suppression was used to deplete endogenous ABIN-2 expression in HeLa S3 cells. Western blotting of cell lysates confirmed that ABIN-2 siRNA, but not an irrelevant control siRNA, significantly reduced steady state levels of ABIN-2 protein (FIG. 7A, upper panels). TPL-2 protein levels were also strikingly reduced by ABIN-2 siRNA treatment. ABIN-2 depletion by RNA interference in 293 cells similarly reduced steady-state levels of TPL-2 protein (FIG. 7B, upper panels). Semi-quantitative PCR demonstrated that ABIN-2 depletion did not alter steady state levels of TPL-2 mRNA in HeLa cells (FIG. 7A, lower panels) or 293 cells (FIG. 7B, lower panels) suggesting that TPL-2 protein levels were down-regulated post-transcriptionally. ABIN-2 depletion did not affect steady-state p105 levels in either HeLa (FIG. 7A) or 293 cells (FIG. 7B). In addition, the ratio of p105/p50 was not affected by ABIN-2 knockdown (FIG. 7C), suggesting that ABIN-2 is not required for constitutive processing of p105 to p50.

The low level of TPL-2 expression in both HeLa and 293 cells prevented its detection after metabolic labeling with [³⁵S]methionine and [³⁵S]cysteine. It was therefore not possible to determine by pulse-chase metabolic labeling whether ABIN-2 knockdown increased TPL-2 turnover. As an alternative approach to investigate whether ABIN-2 binding modulates TPL-2 stability, the effect of ABIN-2-FL binding to TPL-2 was investigated by transient transfection of 293 cells. ABIN-2-FL co-expression significantly increased steady state levels of co-transfected TPL-2 protein compared with EV co-transfected cells (FIG. 7D, upper panels). However, semi-quantitative PCR revealed that ABIN-2-FL did not alter the levels of co-transfected TPL-2 mRNA (FIG. 7D, lower panels). These data suggest that ABIN-2-FL binding stabilizes TPL-2 protein. Consistent with this conclusion, pulse-chase metabolic labeling experiments revealed that ABIN-2-FL increased the half-life of co-transfected TPL-2 (FIG. 7E). Together, the data in this section indicate that TPL-2 must interact with ABIN-2 to maintain TPL-2 metabolic stability.

### Steady-State Levels of ABIN-2 Protein are Substantially Reduced in NF-κB1-Deficient Cells.

TPL-2 protein levels are severely reduced in NF-κB1-deficient cells, as p105 binding is required to maintain the metabolic stability of TPL-2 protein (2, 31). Since the majority of cellular ABIN-2 is associated with p105, the effect of p105 deficiency on steady-state levels of ABIN-2 was investigated. To do this, ABIN-2 was immunoprecipitated from lysates of wild type (nf-κb1^{+/+}) and nf-κb1^{-/-} 3T3 fibroblasts. Western blotting revealed that ABIN-2 was undetectable in the p105-deficient cells, although it was clearly present in wild type cells (FIG. 8A). However, semi-quantitative PCR indicated that ABIN-2 mRNA levels were similar in both cell lines (FIG. 8B). ABIN-2 protein levels were also severely reduced in primary nf-κb1^{-/-} BMDMs compared with nf-κb1^{+/+} control cells (FIG. 8C). Thus, NF-κB1 p105/p50 expression is required to maintain steady-state levels of endogenous ABIN-2 protein. Consequently, lack of TPL-2 expression in nf-κb1^{-/-} cells is likely to be caused by deficiency of both p105/p50 and ABIN-2 (2, 31).

ABIN-2 is not Associated with Active TPL-2 in LPS-Stimulated BMDMs

The observation that the majority of TPL-2 in BMDMs is associated with ABIN-2 (FIG. 4 B and C) suggested that ABIN-2 might function in the TLR4/TPL-2/MEK/ERK signaling pathway (9). It has previously been shown that LPS induces the proteolysis of p105 and the long form of TPL-2 (M1-TPL-2) (8, 31). In initial experiments, the effect of LPS stimulation on ABIN-2 stability was determined in BMDMs. Stimulation with LPS for 60 min induced degradation of a substantial fraction of ABIN-2 (FIG. 9A). LPS activation of endogenous MEK phosphorylation preceded degradation of ABIN-2 (FIG. 9A), suggesting that ABIN-2 proteolysis may be important in downregulation of the signaling pathway. LPS also induced proteolysis of M1-TPL-2 and p105, as expected (8, 31). Pretreatment of cells with the proteasome inhibitor, MG132, blocked LPS-stimulated proteolysis of ABIN-2, M1-TPL-2 and p105 (FIG. 9B). Thus, each of the components of the ABIN-2/TPL-2/p105 ternary complex is proteolysed by the proteasome after LPS stimulation of BMDMs.

LPS stimulation activates the MEK kinase activity of TPL-2 in BMDMs (31), consistent with its essential role in inducing MEK phosphorylation in these cells (9). To investigate whether ABIN-2 is associated with active TPL-2, ABIN-2 was immunoprecipitated from LPS-stimulated BMDMs and the MEK kinase activity of associated TPL-2 determined in a coupled MEK/ERK kinase assay (23). Although large amounts of TPL-2 were present in anti-ABIN-2 antibody immunoprecipitates, no associated MEK kinase was detected with or without LPS stimulation (FIG. 9C). In contrast, substantial MEK kinase activity was evident in anti-TPL-2 immunoprecipitates from lysates of LPS-stimulated cells, as expected (31). Thus, ABIN-2 is not associated with the active pool of TPL-2, suggesting that TPL-2 might dissociate from ABIN-2 after LPS stimulation. To investigate this possibility, lysates of BMDMs were pre-cleared of ABIN-2 by immunoprecipitation and then Western blotted for TPL-2. Similar to previous results (FIG. 4C), minimal ABIN-2-free TPL-2 was detected in unstimulated cells (FIG. 9D). However, LPS stimulation induced a substantial increase of both long and short forms of TPL-2 in the ABIN-2-depleted lysate. Thus, LPS-stimulated activation of TPL-2 (FIG. 9C) correlates with its release from ABIN-2.

No MEK kinase activity was detected in anti-p105 immunoprecipitates from LPS-stimulated BMDM lysates (FIG. 9C), consistent with published data showing that p105 functions as an inhibitor of TPL-2 (2, 31). To determine whether ABIN-2 might also inhibit TPL-2 MEK kinase activity, Myc-TPL-2 was transiently co-expressed with ABIN-2-FL in 293 cells and then isolated by immunoprecipitation with anti-Myc MAb. Myc-TPL-2 MEK kinase activity was not affected by co-expression with ABIN-2-FL (FIG. 9E), although ABIN-2-FL was clearly associated with Myc-TPL-2. In contrast, Ha-p105 co-expression dramatically inhibited Myc-TPL-2 activity, as reported previously (2). Thus ABIN-2 does not appear to function as an inhibitor of TPL-2 MEK kinase activity.

### REFERENCES

1. Beinke, S., M. P. Belich, and S. C. Ley. 2002. The death domain of NF-κB1 p105 is essential for signal-induced p105 proteolysis. J. Biol. Chem. 277:24162-24168.
2. Beinke, S., J. Deka, V. Lang, M. P. Belich, P. A. Walker, S. Howell, S. J. Smerdon, S. J. Gamblin, and S. C. Ley. 2003. NF-κB p105 negatively regulates TPL-2 MEK kinase activity. Mol. Cell. Biol. 23:4739-4752.
3. Belich, M. P., A. Salmeron, L. H. Johnston, and S. C. Ley. 1999. TPL-2 kinase regulates the proteolysis of the NF-κB inhibitory protein NF-κB1 p105. Nature 397:363-368.
4. Beyaert, R., K. Heyninck, and S. van Huffel. 2000. A20 and A20-binding proteins as cellular inhibitors of nuclear factor-KB-dependent gene expression and apoptosis. Biochem. Pharm. 60:1143-1151.
5. Ceci, J. D., C. P. Patriotis, C. Tsatsanis, A. M. Makris, R. Kovatch, D. A. Swing, N. A. Jenkins, P. N. Tsichlis, and N. G. Copeland. 1997. TPL-2 is an oncogenic kinase that is activated by carboxy-terminal truncation. Gene. Devel. 11:688-700.
6. Chang, L., and M. Karin. 2001. Mammalian MAP kinase signalling cascades. Nature 410:37-40.
7. Coope, H. J., P. G. P. Atkinson, B. Huhse, M. P. Belich, J. Janzen, M. Holman, G. G. B. Klaus, L. H. Johnston, and S. C. Ley. 2002. CD40 regulates the processing of NF-κB2 p100 to p52. EMBO J. 21:5375-5385.
8. Donald, R., D. W. Ballard, and J. Hawiger. 1995. Proteolytic processing of NF-κB/IκB in human monocytes. J. Biol. Chem. 270:9-12.
9. Dumitru, C. D., J. D. Ceci, C. Tsatsanis, D. Kontoyiannis, K. Stamatakis, J.-H. Lin, C. Patriotis, N. A. Jenkins, N. G. Copeland, G. Kollias, and P. N. Tsichlis. 2000. TNFα induction by LPS is regulated post-transcriptionally via a TPL2/ERK-dependent pathway. Cell 103:1071-1083.
10. Fries, K. L., W. E. Miller, and N. Raab-Traub. 1999. The A20 protein interacts with the Epstein-Barr virus latent membrane protein 1 (LMP1) and alters the LMP1/TRAF1/TRADD complex. Virology 264:159-166.
11. Ghosh, S., M. J. May, and E. B. Kopp. 1998. NF-κB and Rel proteins: evolutionary conserved mediators of immune responses. Annu. Rev. Immunol. 16:225-260.
12. Heissmeyer, V., D. Krappmann, E. N. Hatada, and C. Scheidereit. 2001. Shared pathways of IκB kinase-induced SCFβTrCP-mediated ubiquitination and degradation for the NF-κB precursor p105 and IκBa. Mol. Cell. Biol. 21:1024-1035.
13. Heyninck, K., M. M. Kreike, and R. Beyaert. 2003. Structure-function analysis of the A20-binding inhibitor of NF-κB activation, ABIN-1. FEBS Lett. 536:135-140.
14. Hochstrasser, D. F., and C. R. Merril. 1988. ‘Catalysts’ for polyacrylamide gel polymerization and detection of proteins by silver staining. Appl. Theor. Electrophor. 1:35-40.
15. Holloway, A. F., S. Rao, and M. F. Shannon. 2001. Regulation of cytokine gene transcription in the immune system. Mol. Imm. 38:567-580.
16. Ishikawa, H., E. Claudio, D. Dambach, C. Raventos-Suarez, C. Ryan, and R. Bravo. 1998. Chronic inflammation and susceptibility to bacterial infections in mice lacking the polypeptide (p) 105 precursor (NF-κB1) but expressing p50. J. Exp. Med. 187:985-996.
17. Kabouridis, P. S., A. I. Magee, and S. C. Ley. 1997. S-acylation of LCK protein tyrosine kinase is essential for its signalling function in T lymphocytes. EMBO J. 16:4983-4998.
18. Karin, M., and Y. Ben-Neriah. 2000. Phosphorylation meets ubiquitination: the control of NF-κB activity. Annu. Rev. Immunol. 18:621-663.
19. Lang, V., J. Janzen, G. Z. Fischer, Y. Soneji, S. Beinke, A. Salmeron, H. Allen, R. T. Hay, Y. Ben-Neriah, and S. C. Ley. 2003. βTrCP-mediated proteolysis of NF-κB1 p105 requires phosphorylation of p105 serines 927 and 932. Mol. Cell. Biol. 23:402-413.
20. Lee, E. G., D. L. Boone, S. Chai, S. L. Libby, M. Chien, J. P. Lodolce, and A. Ma. 2000. Failure to regulate TNF-induced NF-κB and cell death responses in A20-deficient mice. Science 289:2350-2354.
21. Mercurio, F., J. A. DiDonato, C. Rosette, and M. Karin. 1993. p105 and p98 precursor proteins play an active role in NF-κB-mediated signal transduction. Genes. Devel. 7:705-718.
22. Rice, N. R., M. L. MacKichan, and A. Israel. 1992. The precursor of NF-κB p50 has IκB-like functions. Cell 71:243-253.
23. Salmeron, A., T. B. Ahmad, G. W. Carlile, D. Pappin, R. P. Narsimhan, and S. C. Ley. 1996. Activation of MEK-1 and SEK-1 by Tp1-2 proto-oncoprotein, a novel MAP kinase kinase kinase. EMBO J. 15:817-826.
24. Salmeron, A., J. Janzen, Y. Soneji, N. Bump, J. Kamens, H. Allen, and S. C. Ley. 2001. Direct phosphorylation of NF-κB p105 by the IκB kinase complex on serine 927 is essential for signal-induced p105 proteolysis. J. Biol. Chem. 276:22215-22222.
25. Schneider, C., R. A. Newman, D. R. Sutherland, U. Asser, and M. F. Greaves. 1982. A one-step purification of membrane proteins using a high efficiency immunomatrix. J. Biol. Chem. 257:10766-10769.
26. Sha, W. C., H.-C. Liou, E. I. Tuomanen, and D. Baltimore. 1995. Targeted disruption of the p50 subunit of NF-κB leads to multifocal defects in immune responses. Cell 80:321-330.
27. Takeda, K., and S. Akira. 2004. TLR signaling pathways. Sem. Immunol. 16:3-9.
28. Tegethoff, S., J. Behlke, and C. Scheidereit. 2003. Tetrameric oligomerization of IκB kinasse γ (IKKγ) is obligatory for IKK complex activity and NF-κB activation. Mol. Cell. Biol. 23:2029-2041.
29. van Huffel, S., F. Delaei, K Heyninck, D. de Valck, and R. Beyaert. 2001. Identification of a novel A20-binding inhibitor of nuclear factor-KB activation termed ABIN-2. J. Biol. Chem. 276:30216-30223.
30. Warren, M. K., and S, N. Vogel. 1985. Bone marrow-derived macrophages: development and regulation of differentiation markers by colony-stimulating factor and interferons. J. Immunol. 134:982-989.
31. Waterfield, M. R., M. Zhang, L. P. Norman, and S.-C. Sun. 2003. NF-κB1/p105 regulates lipopolysaccharide-stimulated MAP kinase signaling by governing the stability and function of the TPL-2 kinase. Mol. Cell. 11:685-694.
32. Zhang, S. Q., A. Kovalenko, G. Cantarella, and D. Wallach. 2000. Recruitment of the IKK signalosome to the p55 TNF receptor: RIP and A20 bind to NEMO (IKKγ) upon receptor stimulation. Immunity 12:301-311.
33. Zhu, W., J. S. Downey, J. Gu, F. D. Padova, H. Gram, and J. Han. 2000. Regulation of TNF expression by multiple mitogen-activated protein kinase pathways. J. Immunol. 164:6349-6358.

1. A method of using an ABIN2 molecule to stabilize TPL-2 comprising combining an ABIN2 molecule with TPL-2 under conditions wherein said ABIN2 molecule and TPL-2 associate.

2. The method according to claim 1 wherein the ABIN2 molecule comprises residues 1-250 of ABIN2.

3. The method according to claim 1 wherein the ABIN2 molecule comprises residues 194-250 of ABIN2.

4. A method of using an ABIN2 molecule to modulate p105 activity comprising combining an ABIN2 molecule and p105 under conditions wherein said ABIN2 molecule and p105 associate.

5. The method according to claim 4, wherein the ABIN2 molecule comprises residues 194-250 of ABIN2.

6. A method for identifying a compound or compounds capable of modulating the activity of TPL-2, comprising the steps of:
(a) incubating an ABIN2 molecule with the compound or compounds to be assessed; and
(b) identifying those compounds which influence the binding of ABIN2 to TPL-2.

7. A method according to claim 6, wherein the compound or compounds bind to the TPL-2 molecule.

8. A method according to claim 6, wherein the compound or compounds bind to the ABIN2 molecule.

9. A method for identifying a lead compound for a pharmaceutical useful in the treatment of disease, comprising: incubating a compound or compounds to be tested with an ABIN2 molecule, a TPL-2 molecule and a p105 molecule, under conditions in which, but for the presence of the compound or compounds to be tested, ABIN2, TPL-2 and p105 form a ternary complex with a reference affinity;
determining the binding affinity of the ternary complex of ABIN2, TPL-2 and p105 in the presence of the compound or compounds to be tested; and
selecting those compounds which modulate the binding affinity of the ternary complex with respect to the reference binding affinity.

10. A method for identifying a lead compound for a pharmaceutical useful in the treatment of disease, comprising:
incubating a compound or compounds to be tested with an ABIN2 molecule and a TPL-2 molecule, under conditions in which, but for the presence of the compound or compounds to be tested, TPL-2 associates with ABIN2 with a reference affinity;
determining the binding affinity of TPL-2 for ABIN2 in the presence of the compound or compounds to be tested; and
selecting those compounds which modulate the binding affinity of TPL-2 for ABIN2 with respect to the reference binding affinity.

11. A method for identifying a lead compound for a pharmaceutical useful in the treatment of disease, comprising:
incubating a compound or compounds to be tested with an ABIN2 molecule and a p105 molecule, under conditions in which, but for the presence of the compound or compounds to be tested, p105 associates with ABIN2 with a reference affinity;
determining the binding affinity of p105 for ABIN2 in the presence of the compound or compounds to be tested; and
selecting those compounds which modulate the binding affinity of p105 for ABIN2 with respect to the reference binding affinity.

12. A method according to any one of claim 6, 9, 10 or 11, wherein the ABIN2 molecule is defined according to any one of claims 1 to 5.

13. A method according to any one of claims 6, 9, 10 or 11, which is carried out in vivo in a cell.

14. A method according to claim 13, further comprising the measurement of a biological response.

15. A method according to claim 14, wherein said biological response is selected from the group consisting of MEK kinase phosphorylation, MEK kinase activity and ERK kinase activity.

16. A compound identifiable by the method of any one of claims 6, 9, 10, 11 or 15, capable of modulating the direct or indirect interaction of TPL-2 or p105 with ABIN2.

17. A compound according to claim 16, which is an antibody.

18. An antibody according to claim 17, which is specific for ABIN2.

19. A compound according to claim 16, which is a polypeptide.

20. A polypeptide according to claim 19, which is an ABIN2 molecule.

21. A polypeptide according to claim 20, which is a constitutively active mutant or a dominant negative mutant of ABIN2.

22. A method for modulating the activity of p105 and/or TPL-2 in a cell, comprising administering to the cell a compound according to claim 16.

23. A pharmaceutical composition comprising, as active ingredient, a therapeutically effective amount of a compound according to claim 16.

24. A method for treating a condition associated with NFκB induction or repression, comprising administering to a subject a therapeutically effective amount of a compound according to claim 16.

25. A structural model which represents ABIN2 together with TPL-2 and/or p105.

26. A structural model representing a ternary complex comprising ABIN2, p105 and TPL-2.

27. A structural model according to claim 25 or claim 26, which is derived from the atomic coordinates of co-crystallised ABIN2, TPL-2 and p105.

28. A method for preparing a protein crystal comprising TPL-2, comprising the steps of:
preparing a complex comprising a TPL-2 molecule and an ABIN2 molecule; and
crystallising said complex.

29. A method according to claim 28, wherein the complex is a ternary complex which further comprises a p105 molecule.


SEQUENCE LISTING (9 sequences)

SEQ ID NO: 1 - protein, 9 aa, Artificial Sequence
  Synthetic Peptide Epitope
YPYDVPDYA

SEQ ID NO: 2 - DNA, 21 bp, Artificial Sequence
  siRNA antisense
guauuuggccgccgacgcatt

SEQ ID NO: 3 - DNA, 21 bp, Artificial Sequence
  siRNA antisense
ugcgucggcggccaaauactt

SEQ ID NO: 4 - DNA, 36 bp, Artificial Sequence
  synthetic oligonucleotide primer
acgctagtcgactcacctgtacgtcagcttccacgg

SEQ ID NO: 5 - DNA, 34 bp, Artificial Sequence
  synthetic oligonucleotide primer
gcccaggggatccgaatggagtacatgagcaccg

SEQ ID NO: 6 - DNA, 22 bp, Artificial Sequence
  synthetic oligonucleotide primer
ggcggcttggtgactctagata

SEQ ID NO: 7 - DNA, 21 bp, Artificial Sequence
  synthetic oligonucleotide primer
gctcgggcctgctttgaacac

SEQ ID NO: 8 - DNA, 21 bp, Artificial Sequence
  synthetic oligonucleotide primer
ccatgtcgtctggggacgcaa

SEQ ID NO: 9 - DNA, 21 bp, Artificial Sequence
  synthetic oligonucleotide primer
tggcagcactcagacaggtgc